(19) United States
(12) Patent Application Publication
(10) Pub. No.: US 2010/0087869 A1
(43) Pub. Date: Apr. 8, 2010

(54) DEVICES AND METHODS TO LIMIT ABERRANT MOVEMENT OF THE VERTEBRAL BONES

(76) Inventor: M. Samy Abdou, San Diego, CA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/543,410

(22) Filed: Aug. 18, 2009

Related U.S. Application Data
(60) Provisional application No. 61/189,341, filed on Aug. 18, 2008.

(51) Int. Cl. A61B 17/88; A61B 17/70

(52) U.S. Cl. 606/279; 606/246

(57) ABSTRACT

Methods and devices are configured to attach an orthopedic implant onto a first vertebral bone of a functional spinal unit. A segment of the implant forms an abutment surface with a segment of a second vertebral bone within an unstable, or potentially unstable, vertebral column wherein the abutment surface resists and opposes aberrant movement between the first and second vertebral bones within a horizontal plane. The device may be rigidly attached onto the first vertebral bone but movable relative to the second vertebral bone.

### REFERENCE TO PRIORITY DOCUMENT

This application claims priority of co-pending U.S. Provisional Patent Application Ser. No. 61/189,341 filed Aug. 18, 2008. Priority of the aforementioned filing date is hereby claimed and the disclosure of the Provisional Patent Application is hereby incorporated by reference in its entirety.

### BACKGROUND

Progressive constriction of the central canal within the spinal column is a predictable consequence of aging. As the spinal canal narrows, the nerve elements that reside within it become progressively more crowded. Eventually, the canal dimensions become sufficiently small so as to significantly compress the nerve elements and produce pain, weakness, sensory changes, clumsiness and other manifestation of nervous system dysfunction.

Constriction of the canal within the lumbar spine is termed lumbar stenosis. This condition is common in the elderly and causes a significant proportion of the low back pain, lower extremity pain, lower extremity weakness, limitation of mobility and the high disability rates that afflict this age group. With aging and spinal degeneration, displacement of the vertebral bones in the horizontal may occur and the condition is termed Sponylolisthesis. Spondylolisthesis exacerbates the extent of nerve compression within the spinal canal since misalignment of the vertebral bones will further reduce the size of the spinal canal.

Relief for the compressed nerves can be achieved by the surgical removal of the bone and ligamentous structures that constrict the spinal canal. However, decompression of the spinal canal can further weaken the facet joints and increase the possibility of additional aberrant vertebral movement in the horizontal plane. Thus, decompression can worsen the extent of spondylolisthesis or produce spondylolisthesis in an otherwise normally aligned FSU. After decompression, surgeons will commonly fuse and immobilize the adjacent spinal bones in order to prevent the development of post-operative vertebral misalignment and spondylolisthesis.

### SUMMARY

Since fusion will often place additional load on the adjacent spinal segments and hasten degeneration of those levels, it is of significant clinical interest to develop an orthopedic implant that would preventing aberrant movement between adjacent vertebral bones in the horizontal plane while permitting decompression of the nerve elements without concurrent fusion.

Disclosed are methods and devices that are configured to attach an orthopedic implant onto a first vertebral bone of a functional spinal unit. A segment of the implant forms an abutment surface with a segment of a second vertebral bone within an unstable, or potentially unstable, vertebral column wherein the abutment surface resists and opposes aberrant movement between the first and second vertebral bones within a horizontal plane. The device may be rigidly attached onto the first vertebral bone but movable relative to the second vertebral bone.

In one aspect, there is disclosed an orthopedic implant adapted to resist anterior movement between a first vertebral bone and a second vertebral bone in a horizontal plane, comprising: a first member that is adapted to affix onto the first bone; a second member that is adapted to abut a segment of the second bone and that can move relative to the first member; at least one flexible rotational articulation member that is contained within the implant and that provides at least a portion of the movement between the first and second members, the articulation member having: a first hollow cylindrical member comprised of an outer surface with a defined radius from a longitudinal central axis, an inner surface with a defined radius from a longitudinal central axis, a thickness and an internal cavity that is contained within the inner surface, wherein at least one cylindrical tab extends from one end of the first follow cylindrical member wherein the tab circumferentially extends less than one hundred and eighty degrees around the longitudinal central axis; a second hollow cylindrical member comprised of an outer surface with a defined radius from the longitudinal central axis, an inner surface with a defined radius from the longitudinal central axis, a thickness and an internal cavity that is contained within the inner surface of the second hollow cylindrical member, wherein at least one cylindrical tab extends from one end of the second hollow cylindrical member wherein the tab circumferentially extends less than one hundred and eighty degrees around its longitudinal central axis; wherein the first and second members are axially aligned and wherein one tab of the first member is positioned within the internal cavity of the second member and one tab of the second member is positioned within the internal cavity of the first member; wherein at least one flexible element connects the inner surface of the tab member of the first member with the inner surface of the second member and at least one flexible element connects the inner surface of the tab member of the second member with the inner surface of the first member so that said first and second may smoothly rotate relative to one another about the central longitudinal axis.

In another aspect, there is disclosed an orthopedic implant adapted to resist anterior movement between a first vertebral bone and a second vertebral bone in a horizontal plane, comprising: a first member that is adapted to affix onto the first bone, wherein the first member contains a cavity that is adapted to contain a bone graft material and fuse with the first bone; a second member that is adapted to abut a segment of the second bone, but not rigidly affix onto it; wherein the implant permits relative movement between the first and second vertebral bones.

In another aspect, there is disclosed a method for resisting translation of a first vertebral bone relative to a second vertebral bone in a horizontal plane, comprising: rigidly affixing an implant onto the first bone, wherein the implant contains a cavity that is adapted to contain a bone graft material and to fuse with the first bone; placing a segment of the implant posterior to a posterior surface of the second vertebral bone; and positioning the implant segment so that it abuts, but does not rigidly affix, onto the posterior surface of the second vertebral bone; wherein the implant permits relative movement between the first and second vertebral bones.

Other features and advantages should be apparent from the following description of various embodiments, which illustrate, by way of example, the principles of the disclosed devices and methods.

### BRIEF DESCRIPTION OF THE DRAWINGS

FIG. 1 shows a diagrammatic representation of a spinal vertebral bone in multiple views.

FIGS. 2A and 2B illustrate a functional spinal unit (FSU).

FIG. 3A illustrates three vertebral bones with relatively normal alignment

FIG. 3B shows the anterior displacement of the middle bone relative to the inferior-most bone.

FIG. 3C shows an inferior vertebral bone of a functional spinal unit (FSU) in the horizontal plane.

FIG. 3D illustrates the vertebral bone of FIG. 3C in the vertical plane.

FIG. 4 illustrates perspective views of a first device embodiment.

FIG. 5 shows the device of FIG. 4 views in multiple orthogonal planes.

FIG. 6 shows exploded views of the device of FIG. 4.

FIG. 7A shows a proposed site of resection (lines R).

FIG. 7B shows the FSU after bone removal.

FIG. 8 shows the anterior aspect of the device when implanted.

FIGS. 9A, 9B and 10 show an alternate embodiment of a device.

FIG. 11A shows an intact spinous process.

FIG. 11B shows a vertebral segment S at the base of a removed spinous process.

FIG. 12 shows device 205 implanted onto a functional spinal unit.

FIGS. 13 and 13B show an alternate device embodiment.

FIGS. 14A and 14B show the alternate device attached to a lamina.

FIG. 15 shows an alternate embodiment of a device.

FIG. 16A shows the device of FIG. 15 in an exploded state.

FIG. 16B shows a spherical member of the device of FIG. 15.

FIG. 16C shows a cross-sectional view through a rod and sphere of the device of FIG. 15.

FIGS. 17 and 18 show the device of FIG. 15 attached to a spine model.

FIGS. 19, 20A and 20B illustrates a different method of use for the device of FIG. 15.

FIGS. 21A and 21B show an additional method of use of the device of FIG. 15.

FIG. 22 shows another embodiment of a device.

FIG. 23 shows the device of FIG. 22 in an exploded state.

FIGS. 24 through 29B show various view of a pivot member and its components.

FIGS. 28A through 29B illustrate the deformation of internal flat crossed slats with movement of the pivot member to either rotational extreme.

FIG. 30 shows an implanted device.

FIGS. 31-33 show views of an alternate embodiment of a device.

FIGS. 34 and 35 show the device of FIGS. 31-33 in an implanted state.

FIG. 36 illustrates an embodiment wherein two of the devices shown in FIG. 34 are connected across the vertebral midline.

FIGS. 37A and 37B show multiple views of an additional embodiment of a device.

FIG. 38 shows the device of FIG. 37A anchored to the spine.

FIG. 39A shows the lateral aspect of the spine.

FIG. 39B shows another view of the device of FIG. 37A.

FIGS. 40A and 40B show another embodiment of a device.

FIG. 41 shows the device of FIGS. 40A-40B being implanted.

FIG. 42 illustrates the device of FIGS. 40A-40B implanted on each side of the vertebral midline and preventing anterior spondylolisthesis of L4 relative to L5.

FIGS. 43-45 illustrate another embodiment of a device.

### DETAILED DESCRIPTION

In order to promote an understanding of the principals of the invention, reference is made to the drawings and the embodiments illustrated therein. Nevertheless, it will be understood that the drawings are illustrative and no limitation of the scope of the invention is thereby intended. Any such alterations and further modifications in the illustrated embodiments, and any such further applications of the principles of the invention as illustrated herein are contemplated as would normally occur to one of ordinary skill in the art.

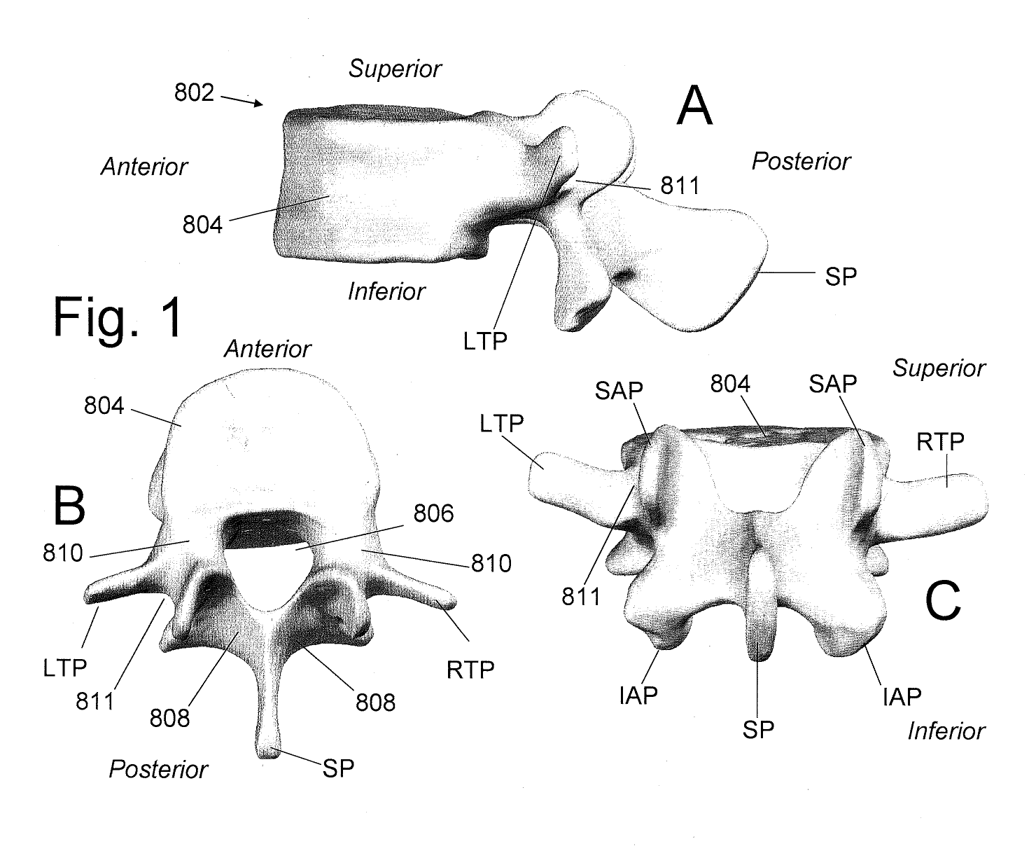

FIG. 1 shows a diagrammatic representation of a spinal vertebral bone 802 in multiple views. For clarity of illustration, the vertebral bone of FIG. 1 and those of other illustrations presented in this application are represented schematically and those skilled in the art will appreciate that actual vertebral bodies may include anatomical details that are not shown in these figures. Further, it is understood that the vertebral bones at a given level of the spinal column of a human or animal subject will contain anatomical features that may not be present at other levels of the same spinal column. The illustrated vertebral bones are intended to generically represent vertebral bones at any spinal level without limitation. Thus, the disclosed devices and methods may be applied at any applicable spinal level.

Vertebral bone 802 contains an anteriorly-placed vertebral body 804, a centrally placed spinal canal and 806 and posteriorly-placed lamina 808. The pedicle (810) segments of vertebral bone 802 form the lateral aspect of the spinal canal and connect the laminas 808 to the vertebral body 804. The spinal canal contains neural structures such as the spinal cord and/or nerves. A midline protrusion termed the spinous process (SP) extends posteriorly from the medial aspect of laminas 808. A protrusion extends laterally from each side of the posterior aspect of the vertebral bone and is termed the transverse process (TP). A right transverse process (RTP) extends to the right and a left transverse process (LTP) extends to the left. A superior protrusion extends superiorly above the lamina on each side of the vertebral midline and is termed the superior articulating process (SAP). An inferior protrusion extends inferiorly below the lamina on each side of the vertebral midline and is termed the inferior articulating process (IAP). Note that the posterior aspect of the pedicle can be accessed at an indentation 811 in the vertebral bone between the lateral aspect of the SAP and the medial aspect of the transverse process (TP). In surgery, it is common practice to anchor a bone fastener into the pedicle portion of a vertebral bone by inserting the fastener through indentation 811 and into the underlying pedicle.

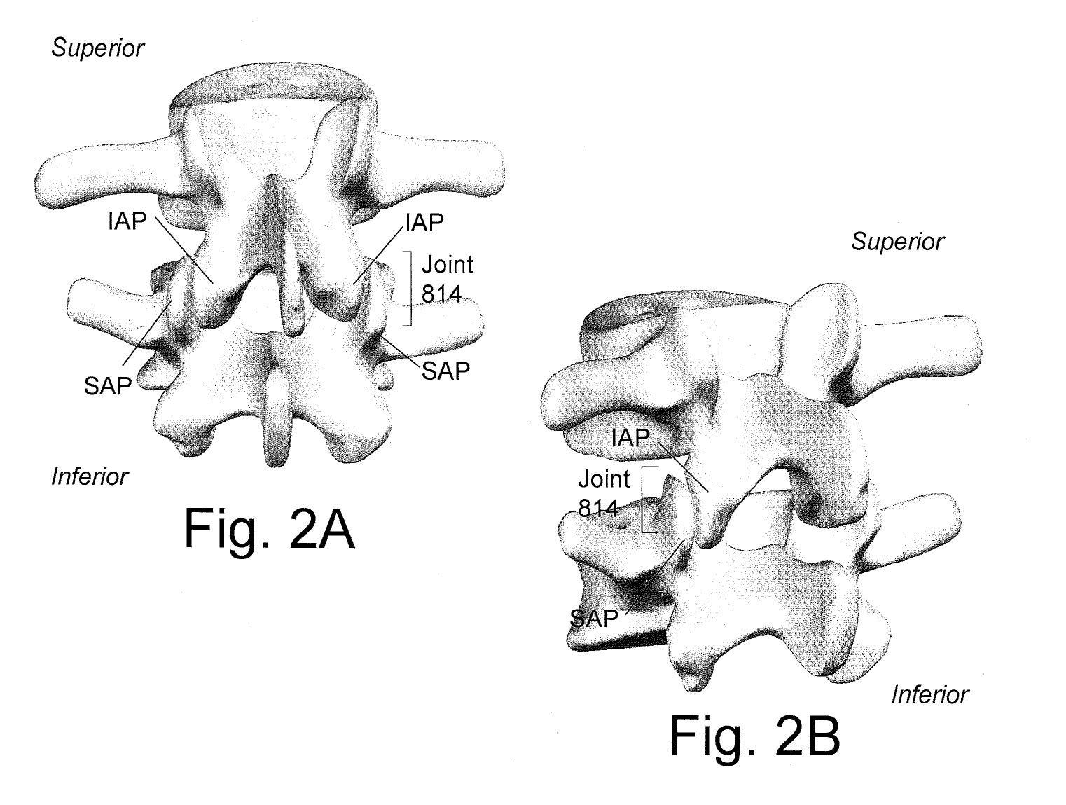

FIGS. 2A and 2B illustrate a functional spinal unit (FSU), which included of two adjacent vertebrae and the intervertebral disc between them. The intervertebral disc resides between the inferior surface of the upper vertebral body and the superior surface of the lower vertebral body. (Note that a space is shown in FIGS. 2A and 2B where intervertebral disc would reside.) FIG. 2A shows the posterior surface of the adjacent vertebrae and the articulations between them while FIG. 2B shows an oblique view. Note that FSU contains a three joint complex between the two vertebral bones, with the intervertebral disc comprising the anterior joint. The posterior joints include a facet joint 814 on each side of the midline, wherein the facet joint contains the articulation between the IAP of the superior vertebral bone and the SAP of the inferior bone.

The preceding illustrations and definitions of anatomical structures are known to those of ordinary skill in the art. They are illustrated in more detail in Atlas of Human Anatomy, by Frank Netter, third edition, Icon Learning Systems, Teterboro, N.J. The text is hereby incorporated by reference in its entirety.

In the functional spinal unit, a substantial portion (up to 80%) of the vertical load is borne by the intervertebral disc and the anterior column. (The term “vertical load” refers to the load transmitted in the vertical plane through the erect human spine. The “anterior column” is used here to designate that portion of the vertebral body and/or FSU that is situated anterior to the posterior longitudinal ligament and includes the posterior longitudinal ligament. Thus, its use in this application encompasses both the anterior and middle column of Denis. See The three column spine and its significance in the classification of acute thoracolumbar spinal injuries. By Denis, F. Spine 1983 November-December; 8(8):817-31. The article is incorporated by reference in its entirety.) Conversely, a substantial portion of load transmitted through the functional spine unit in the horizontal plane is borne by the facet joint and the posterior column. (The “posterior column” is used here to designate that portion of the vertebral body and/or FSU that is situated posterior to the posterior longitudinal ligament.) Generally, the forces acting in the horizontal plane are aligned to cause an anterior displacement of the superior vertebral body relative to the inferior vertebral body of a functional spinal unit. These forces are counteracted by the facet joints which are formed by the abutment surfaces of the IAP of the superior vertebral bone and the SAP of the inferior bone.

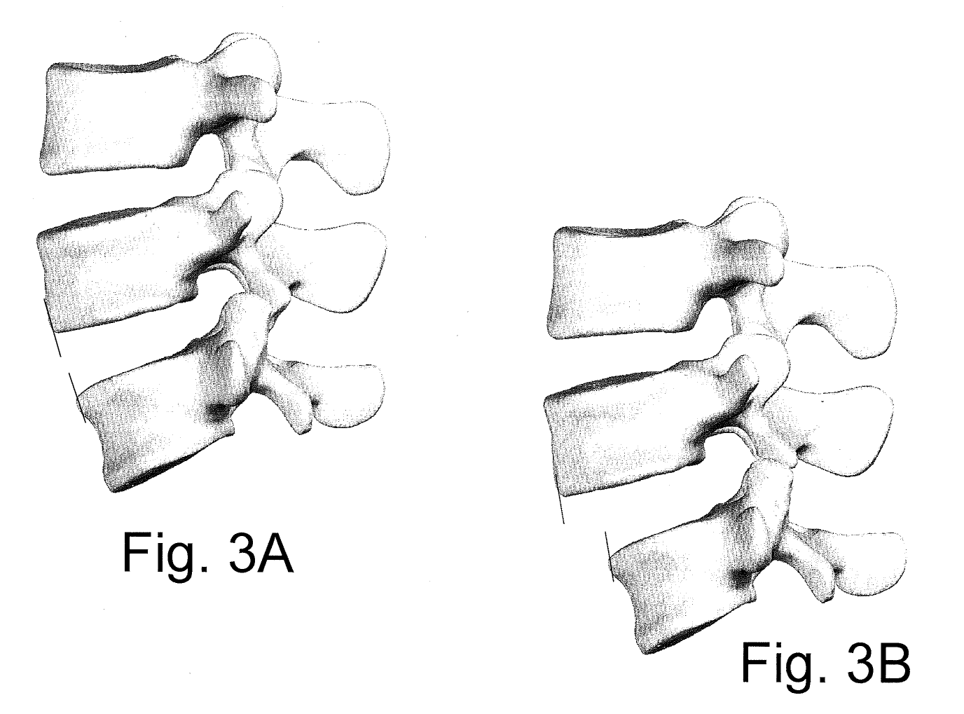

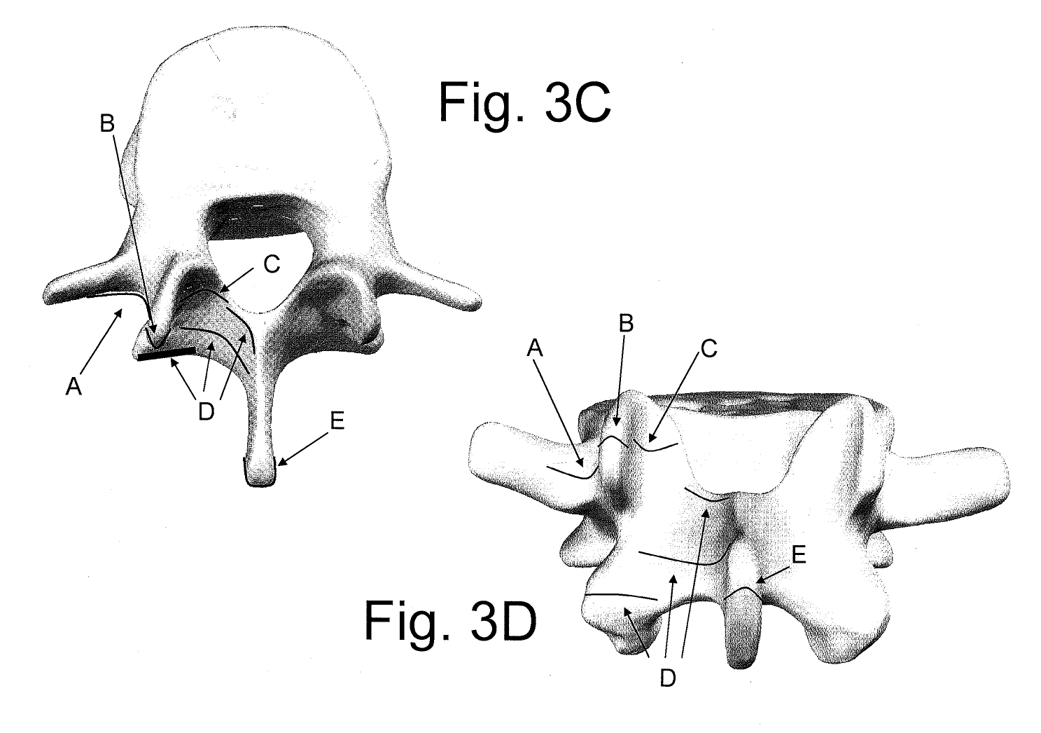

In a healthy spine functioning within physiological parameters, the two facet joints of an FSU collectively function to prevent aberrant relative movement of the vertebral bones in the horizontal plane. With aging and spinal degeneration, displacement of the vertebral bones in the horizontal may occur and the condition is termed Sponylolisthesis. FIG. 3A illustrates three vertebral bones with relatively normal alignment, whereas FIG. 3B shows the anterior displacement of the middle bone relative to the inferior-most bone. In the illustration, the vertebral column of FIG. 3B is said to have an anterior spondylolisthesis of the middle vertebral bone relative to the inferior-most vertebral bone, since the middle bone is anteriorly displaced relative to the inferior bone.

A spondylolisthesis can be anterior, as shown in FIG. 3B, or posterior wherein a superior vertebral bone of a functional spinal unit is posteriorly displaced in the horizontal plane relative to the inferior vertebral bone. Anterior Sponylolisthesis is more common and more clinically relevant than posterior Sponylolisthesis. (Sponylolisthesis can be further classified based on the extent of vertebral displacement. See Principles and practice of spine surgery by Vaccaro, Bets, Zeidman; Mosby press, Philadelphia, Pa.; 2003. The text is incorporated by reference in its entirety.)

With degeneration of the spine, constriction of the spinal canal (spinal stenosis) and impingement of the contained nerve elements frequently occurs and is termed spinal stenosis. Spondylolisthesis exacerbates the extent of nerve compression within the spinal canal since misalignment of bone within the horizontal plane will further reduce the size of the spinal canal. Relief for the compressed nerves can be achieved by the surgical removal of the bone and ligamentous structures that constrict the spinal canal. However, decompression of the spinal canal can further weaken the facet joints and increase the possibility of additional aberrant vertebral movement in the horizontal plane and worsen the extent of spondylolisthesis or produce spondylolisthesis in an otherwise normally aligned FSU. After decompression, surgeons will commonly fuse and immobilize the adjacent spinal bones in order to prevent the development of post-operative vertebral misalignment and spondylolisthesis.

Disclosed are methods and devices configured to attach an orthopedic implant onto a first vertebral bone of a functional spinal unit. A segment of the device would form an abutment surface with a segment of a second vertebral bone within an unstable, or potentially unstable, vertebral column wherein the abutment surface would resist aberrant movement between the first and second vertebral bones within the horizontal plane. In an embodiment, the device forms an osseous or bony bond with the first vertebra. In an embodiment, the device contains a cavity into which bone graft material (i.e., a material adapted to form bone such as bone fragments, synthetic bone graft substitutes, growth factors that are capable of promoting and forming bone, and the like) is placed in order to form a bone fusion mass within the cavity, wherein the mass is also fused with the first vertebral bone. In an embodiment, the device also contains a surface that can directly fuse onto the first vertebral bone. (For example, a device surface may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening.).

The abutment surface may be positioned to effectively oppose the undesired movement in the horizontal plane. For example, if anterior spondylolisthesis is to be resisted, it is advantageous to attach the device to a superior vertebra and position the abutment surface of the device posterior to a posterior surface of an inferior vertebra. Alternately, the abutment surface may be positioned posterior to a second implant that is attached to the second vertebra, wherein an abutment is formed between an abutment surface of each of the two implants. In order to prevent posterior displacement of a superior vertebral bone relative to an inferior vertebral bone, the device is attached to the inferior vertebral bone and positioned to abut a posterior surface of the superior vertebra. In order to prevent lateral displacement of a first vertebral bone relative to a second vertebral bone, the device is attached onto a lateral surface (such a the lateral aspect of the vertebral body) of a first vertebral bone and forms an abutment surface with a lateral surface of a second vertebral bone. Depending on the direction of the lateral aberrant movement it is designed to prevent, the implant may be attached to the superior vertebra and abut the inferior vertebral bone or visa versa. Since anterior spondylolisthesis is clinically the most common aberrant movement in the horizontal plane, the drawings and the embodiments of the devices illustrated herein are described while in use to prevent anterior spondylolisthesis. However, it should be clearly understood that each of the devices and/or methods disclosed herein can be alternatively used to prevent aberrant horizontal vertebral movement in any direction.

The devices illustrated herein are adapted to rigidly attach onto a first vertebral bone and provide an abutment surface with a second vertebral bone. In general, the device is not rigidly attached to the second vertebral bone. There is permitted at lease some movement between the first and second vertebral bones, while effectively limiting aberrant vertebral movement between the two bones in horizontal plane.

FIG. 3C shows an inferior vertebral bone of a functional spinal unit (FSU) in the horizontal plane and FIG. 3D illustrates the same vertebral bone in the vertical plane, wherein the posterior surface of the vertebral bone is demonstrated. Lines A through E illustrate segments of the posterior surface of the inferior vertebral bone against which an implant will be positioned so as to resist forward displacement of the superior vertebral bone in the horizontal plane. Pursuant to this disclosure, an implant is rigidly affixed to the superior vertebral bone of an FSU and a segment of that implant abuts the inferior vertebral bone at one or more of the regions depicted by Lines A through E. In this way, the implant resists aberrant anterior movement (that can form an anterior spondylolisthesis) of the superior vertebral bone relative to the inferior vertebral bone in horizontal plane. It is understood that an implant could be similarly used to resist aberrant posterior movement (that can form a posterior spondylolisthesis) of the superior vertebral bone relative to the inferior vertebral bone in horizontal plane by affixing the implant to the inferior vertebra and positioning the implant to abut the superior vertebra. It is further understood that all devices and methods recited in the following disclosure can be similarly re-configured to resist the formation and/or progression of a posterior spondylolisthesis.

Lines A show the depression between the lateral aspect of the SAP and the transverse process (this region contains region 811 of FIG. 1B). Lines B show the protrusion formed by the posterior aspect of the SAP. Lines C refer to the depression formed within the medial aspect of the SAP (and lateral lamina). Lines D refer to the posterior aspect of the lamina and/or posterior aspect of the IAP. Lines E refer to the protrusion formed by the posterior aspect of the spinous process. Use of each of these regions, alone or in combination, as a abutment surface for an implant attached to the superior vertebra is an integral feature of the disclosed invention.

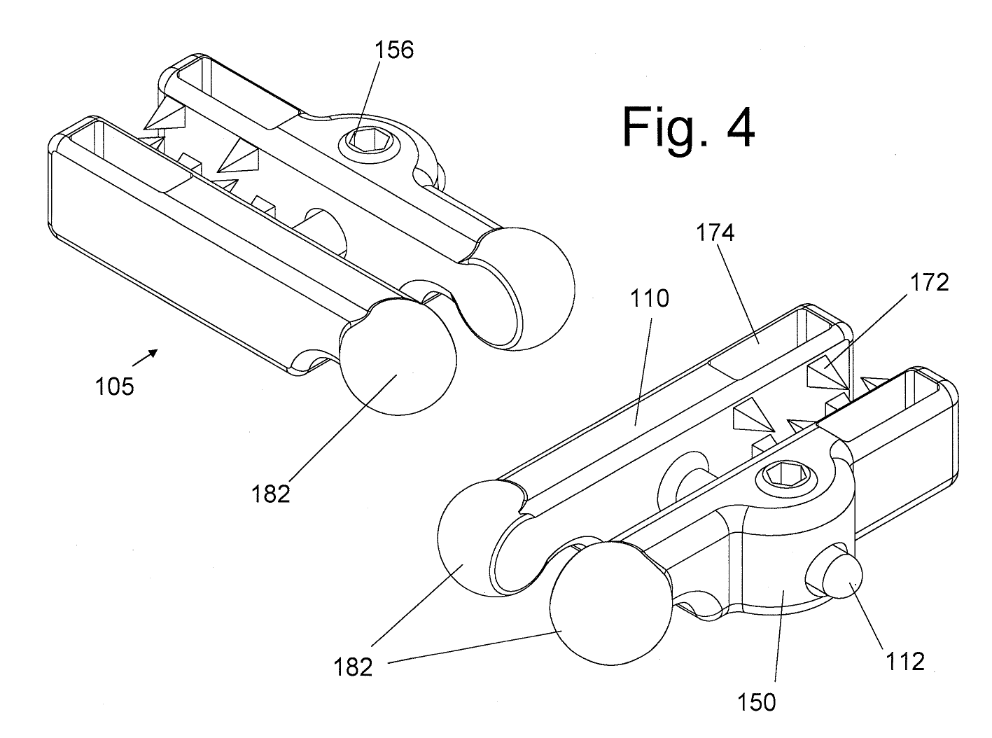

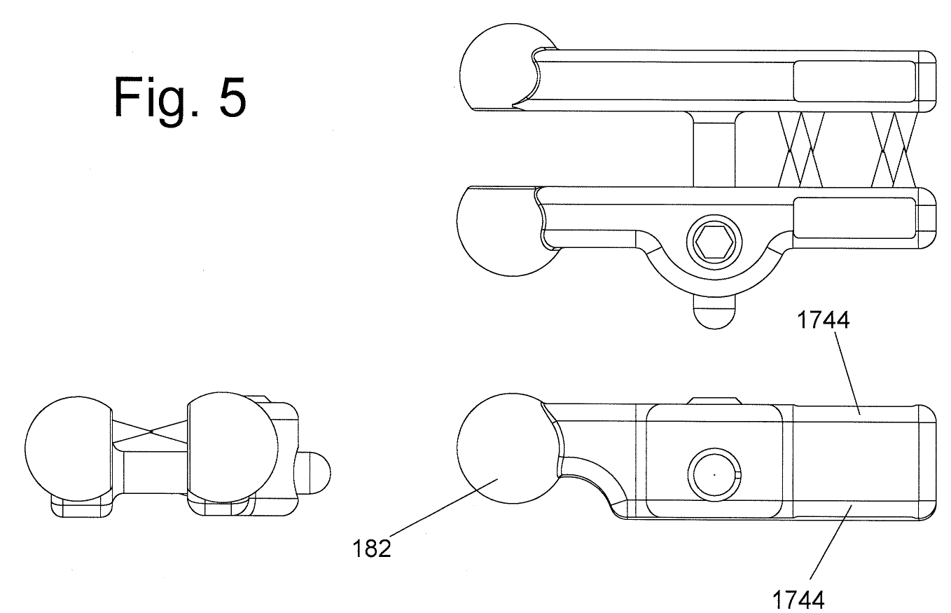

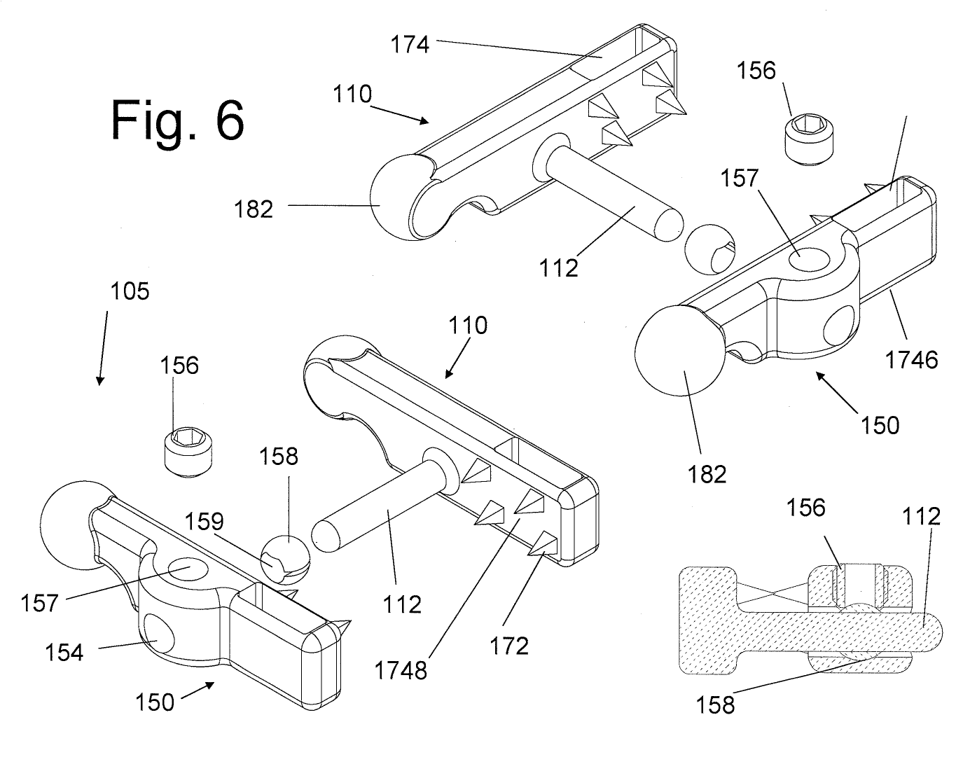

FIG. 4 illustrates perspective views of a first device embodiment. FIG. 5 shows device views in multiple orthogonal planes. An illustration of the disassembled device is shown in FIG. 6.

Device 105 is comprised of member 110 and 150. Bar 112 rigidly extends from the medial surface of member 110 and is disposed within bore 154 of member 150. A threaded set screw 156 (threads not shown) is situated within threaded bore 157 (threads not shown) of member 150 and contains a hex drive within the superior surface that is adapted to accepted a hex screw driver. Bore 157 communicates with bore 154 within member 110, such that advancement of the set screw 156 will cause compression of bar 112 and immobilization of the member 110 relative to member 150 (see the sectional view of FIG. 6). Protrusion 112 is contained within central bore 159 of split spherical member 158 when in bore 154. This permits the adjustment of the relative angle between members 110 and 150.

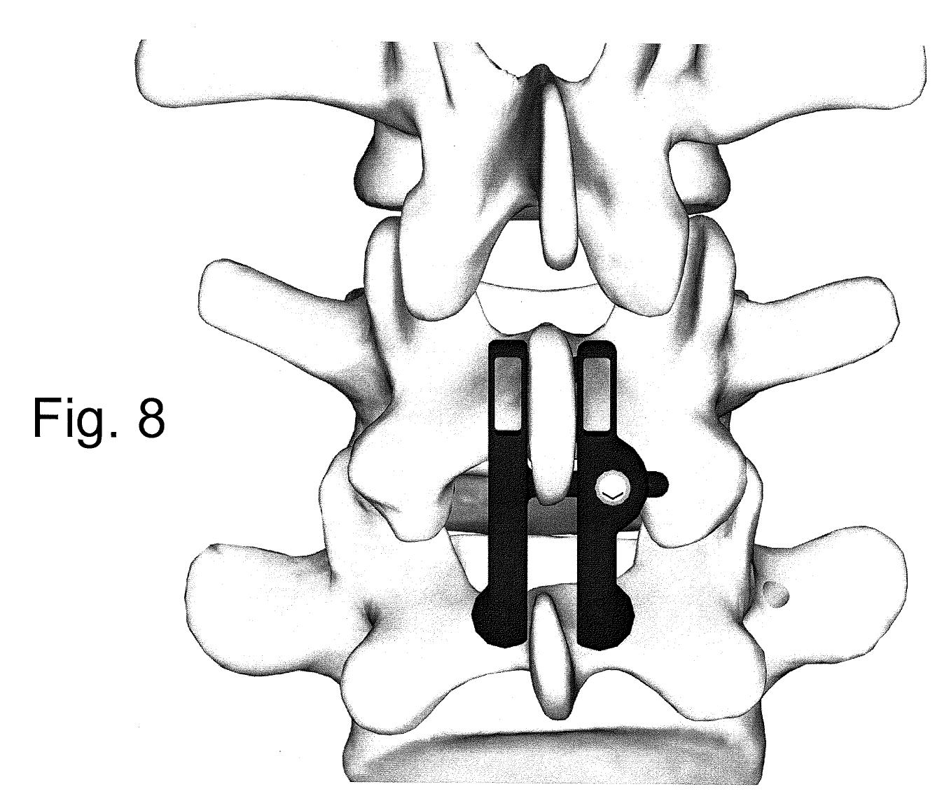

Each of members 110 and 150 contain pointed protrusions 172 that are adapted to engage a bone surface of a first vertebra and anchor the device to it. In an embodiment, at least one of members 110 and 150 contains a compartment 174 adapted to house a bone graft or bone graft substitute that functions to fuse the device onto the first bone. The compartment has an upper opening 1744 and lower opening 1746 that permit communication between the compartment and the outer aspect of the device. A first opening 1744 is used to place the bone-forming material into compartment 1744. Opening 1746 is located on the opposing side of compartment 174 (that is, the anterior aspect of the device when implanted as shown in FIG. 8) so that the bone-forming material within compartment 174 can fuse with the posterior aspect of the underlying lamina. In addition (or alternatively to opening 1746), side holes (not shown) may be placed within the medial wall of compartment 174 at or about region 1748 so that the bone-forming material can fuse with the side of the spinous process. A second end of each of members 110 and 150 contains a protrusion 182 that is adapted to abut against the posterior aspect of the lamina of a second vertebra.

In a preferred embodiment, the device is placed with device 110 rigidly affixed onto an upper vertebra and protrusions 182 abutting a lower vertebra so that anterior movement of the upper vertebra relative to the lower vertebra (and spondylolisthesis formation or progression) is prevented. The device is shown attached to a vertebral model in FIG. 4. For clarity of illustration, the spine is represented schematically and those skilled in the art will appreciate that an actual spine may include anatomical details not shown in FIG. 4.

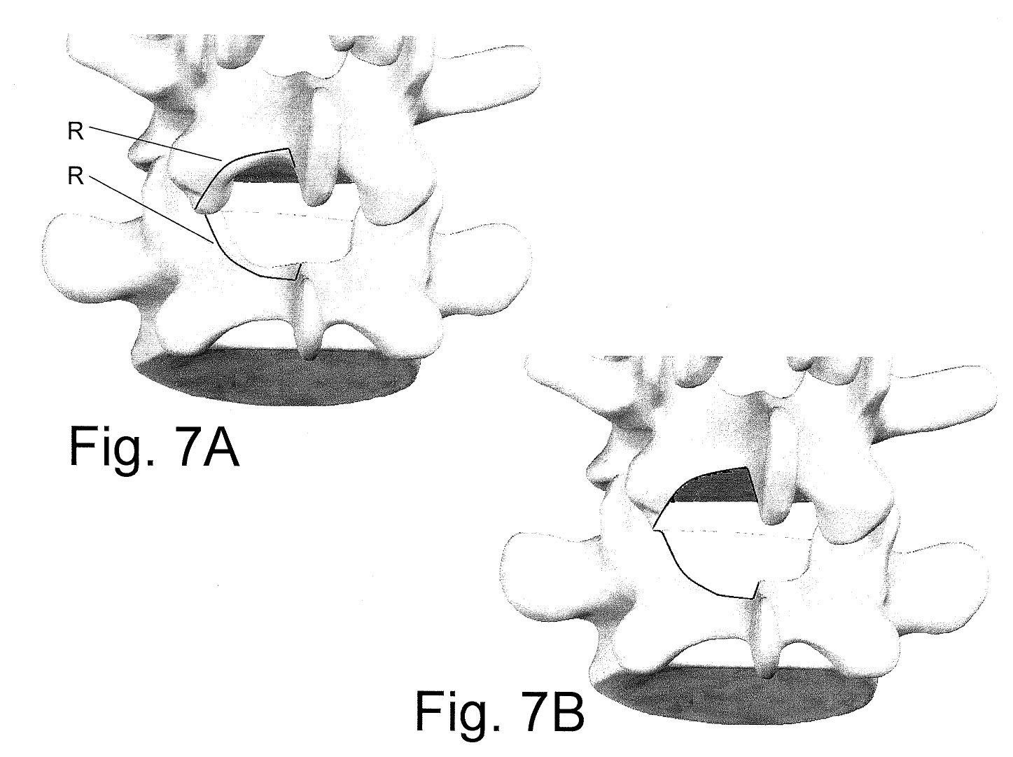

A method of use is herein disclosed. The spinal level that will be implanted is selected by the surgeon. With the patient preferably positioned supine, the spine is approached from a posterior approach so the posterior aspect of the spinal segment that will be implanted is reached. A decompression of the nerve elements may or may not be performed prior to device implantation. In a preferred embodiment, a decompression is performed wherein the substantial portion of the lamina of the superior and inferior vertebral bones is preserved. This may be accomplished by removing the medial aspect of at least one of the two facet joints at the implantation level, wherein the medial aspect of the IAP of the superior vertebral bone and the medial aspect of the SAP of the inferior bone is removed (FIG. 7A shows the proposed site of resection (lines R) while FIG. 7B shows the FSU after bone removal. Note that the illustration also shows removal of a small portion of the lamina of the superior and inferior vertebral bones. In addition, note the diminished portion of facet joint that is left after resection). Preferably, but not necessarily, the ligamentum flavum between the lamina of the superior and inferior vertebral bones is also removed. This provides decompression of the nerve elements at the implantation level but may concurrently weaken the resistance to aberrant movement in the horizontal plane and may lead to spondylolisthesis formation. The interspinous ligament may or may not be removed. While the decompression is illustrated on one side of the vertebral midline in FIG. 7b, it may be also performed bilaterally.

The lateral aspects of the spinous process and/or the posterior aspect of the lamina of the superior vertebral bone are abraded or embedded with shallow cuts in order to decorticate the bone surface and encourage fusion mass formation. The device 105 is positioned with member 110 and member 150 on opposite sides of the spinous processes of each of the superior and inferior vertebral bones. The device is moved until protrusion 182 of each member 110 and 150 abuts the posterior aspect of the lamina of the inferior vertebral bone. With protrusions 182 held in position, a pliers-like compression device (not shown) is used to forcibly compress and drive members 110 and 150 towards one another. Spiked protrusions 172 are forcefully driven into each side of the spinous process of the superior vertebral bone. Set screw 156 is then advanced so as to lock members 110 and 150 relative to one another and immobilize device 105 relative to the superior vertebral bone. Cavity 174 is then packed with bone graft material through upper opening 1744. The bone graft material may be forced through the lower opening 1746 and onto the lamina below. The bone graft material may also make contact with the bony side surface of the spinous process which device 105 is attached (assuming the device contains cut outs of the medial wall of cavity 174 at, or about, region 1748).

Device 105 and the method of use disclosed above will then provide a bony attachment with the superior vertebral bone through the fusion mass contained in cavity 174. (Alternatively, the device surfaces that contact the superior vertebral bone (but not the inferior vertebral bone) may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening). The device provides an abutment surface (protrusions 182) against the posterior surface of the lamina of the inferior vertebral bone. In this way, device 105 permits continued motion between both vertebral bones while resisting the formation or progression of an anterior spondylolisthsis. Since the lamina at all vertebral level is angled so as to extend from a more anterior superior edge to a more posterior inferior edge, device 105 will provide resistance to anterior spondylolisthesis and limit the extent of extension of the superior vertebral bone relative to the inferior vertebral bone.

In an alternative method of implantation, device 105 may be rigidly affixed to the spinous process of an inferior vertebral bone and protrusions 182 positioned to abut the lamina of a superior vertebral bone. When implanted in this manner, the device is configured to resist posterior spondylolisthesis of the superior vertebral bone relative to the inferior vertebral bone.

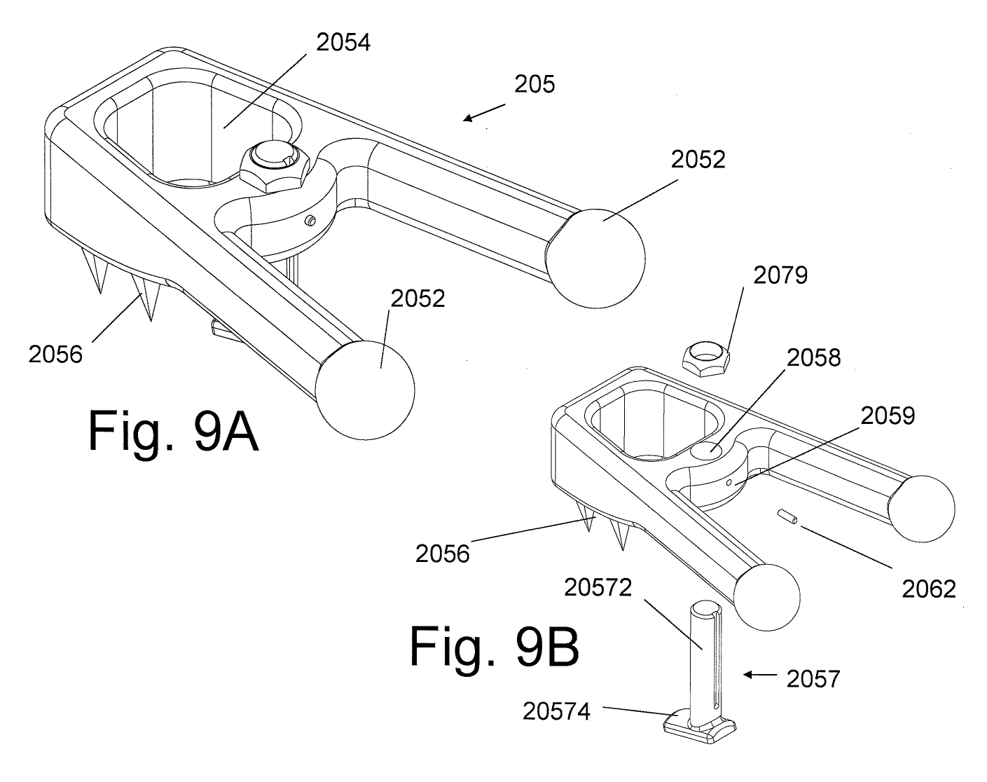

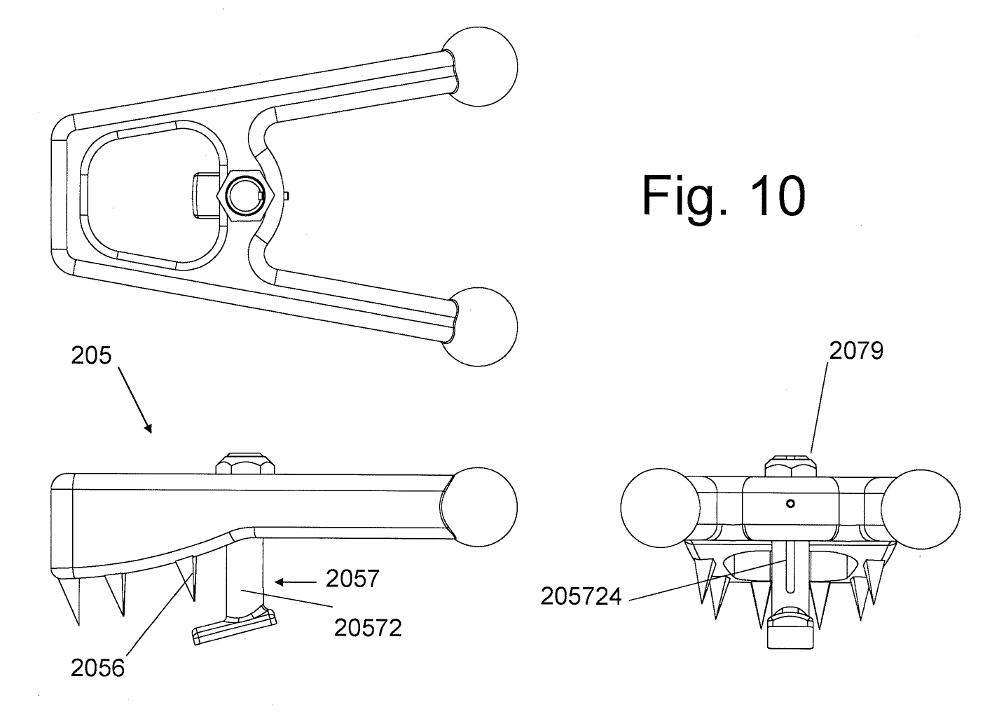

An alternative embodiment is illustrated in FIGS. 9 to 13. The device 205 is adapted to attach and fuse onto the lamina portion of a first vertebra after the removal of at least a portion of the spinous process. Protrusions 2052 are adapted to abut the posterior aspect of the lamina of a second vertebra and prevent aberrant displacement in the horizontal plan of the first vertebra relative to the second vertebra. Preferably, the device contains a cavity 2054 that is adapted to house a bone-forming material. As shown, the device is formed of an “A” shaped frame with cavity 2054 and protrusions 2052. The inferior surface contains protrusions 2056. Bore hole 2058 is adapted to house member 2057. Bore 2058 extends from an upper surface of the device to a lower surface. An additional side bore hole 2059 is adapted to contain pin 2062—which prevents rotation of member 2057 relative to bore hole 2058.

Member 2057 has a threaded post 20572 (threads not shown) and a foot member 20574 that collective function as a hook that is adapted to rest against the anterior aspect of the lamina (that is, that portion of the lamina that faces the spinal canal). Threaded nut 2079 (threads not shown) is adapted to interact with the threads of threaded post 20572 so that rotation of nut 2079 can cause foot member 20574 to move towards or away form the body of device 205. A channel 205724 is disposed in post 20572 and adapted to accept pin 2062. Once again, pin 2062, when positioned through bore hole 2059 and into channel 205724 prevents rotation of post 20572 relative to bore 2058.

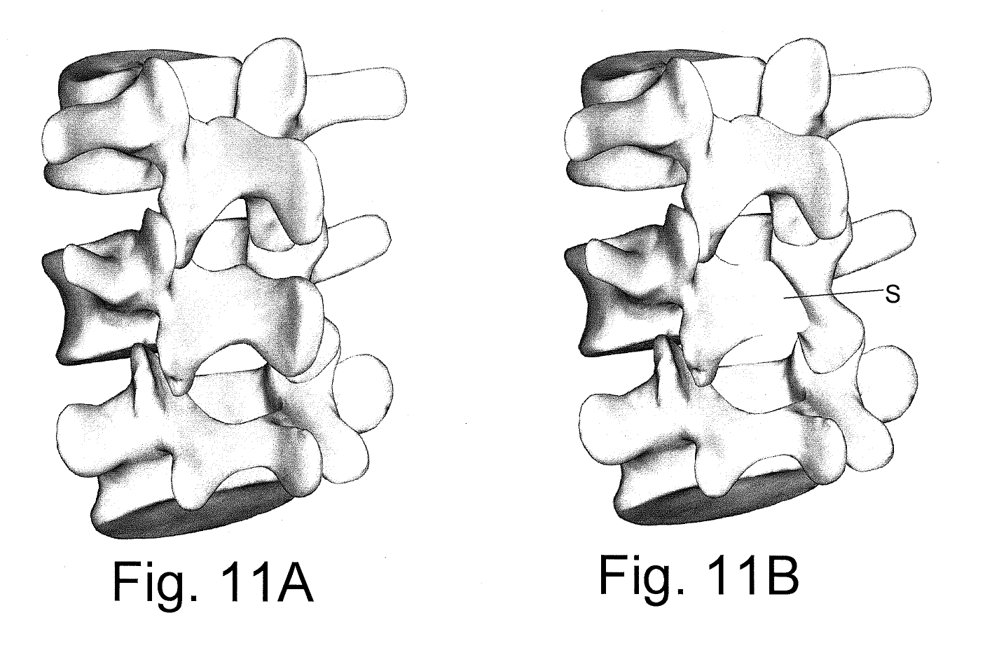

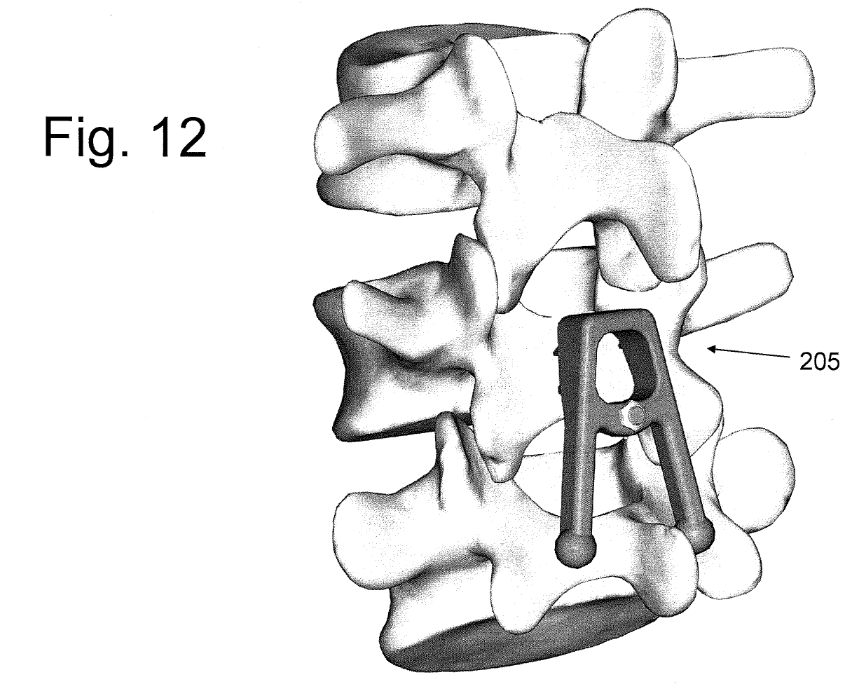

An intact spinous process is shown in FIG. 11A, whereas a vertebral segment S at the base of a removed spinous process is shown in FIG. 11B. In order to implant device 205, the spinous process (or portion thereof) of the superior vertebral bone is removed and posterior surface of the lamina is abraded or cut lightly in order to decorticate the bone in preparation for bone fusion. Device 205 is positioned with cavity 2054 posterior to the cut surface of the spinous process. Protrusions 2052 are positioned to abut the posterior aspect of the lamina of the inferior vertebral bone. Member 2057 is positioned with foot member 20574 along the anterior/inferior surface of the lamina of the superior vertebra. Spiked protrusions 2056 are forced into the posterior aspect of the lamina and nut 2079 is advanced until the lamina in captured between the anterior foot member 20574 and the posterior spiked member 2056. Through out the implantation, protrusions 2052 are maintained in contact with the posterior aspect of the lamina of the inferior vertebra. Cavity 2054 is then filled with bone graft material in order to form a bony fusion between the cavity contents and the superior vertebral bone. FIG. 12 shows device 205 implanted onto a functional spinal unit.

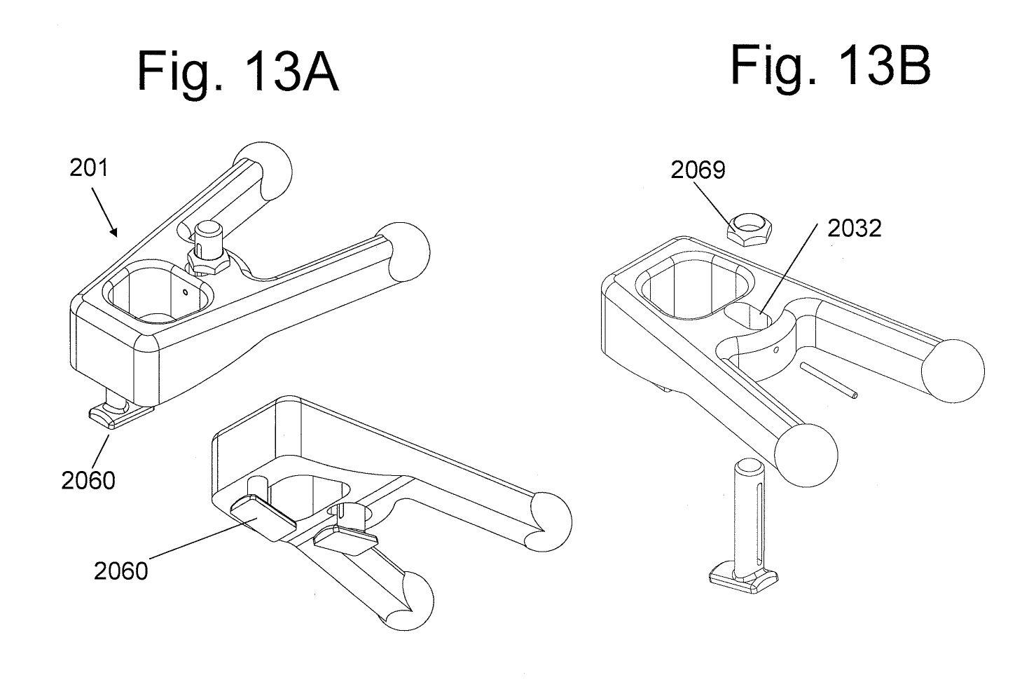

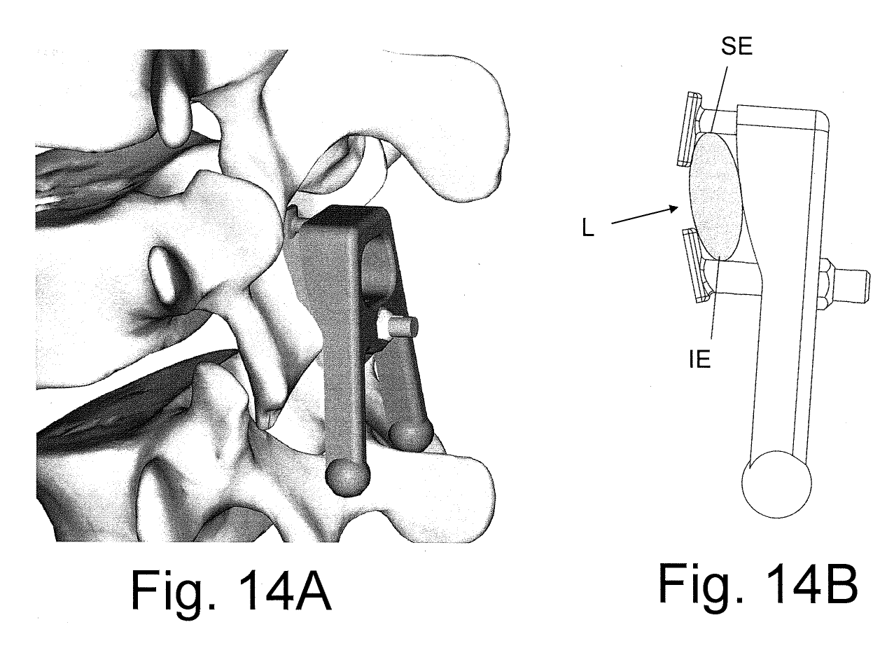

An additional device embodiment is shown in FIGS. 13A through 14B. While similar to device 205, device 201 contains a second hook member 2060 and a slot 2032 that is adapted to contain member 2057 and allow its translation. Spike protrusions 2056 may or may not be present (shown not present). At implantation, device 201 attaches onto the superior edge (SE) and inferior edges (IE) of the lamina (L) and the superior (2060) and inferior (2057) hook members are used to clamp the lamina. After the hook members have encircled the lamina, locking screw 2069 is used to tightly capture the lamina with hook member 2057. The device is now rigidly affixed to the lamina in FIG. 14A and schematically shown attached to a lamina in FIG. 14B. As in prior embodiments, a cavity is contained within the device that is adapted to contain a bone graft material.

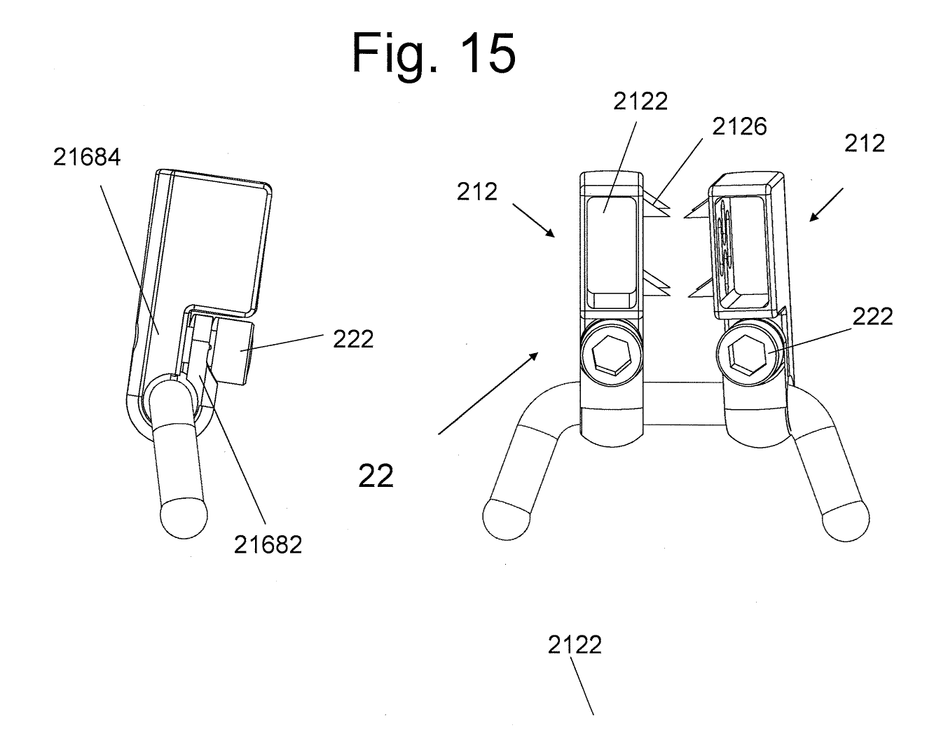

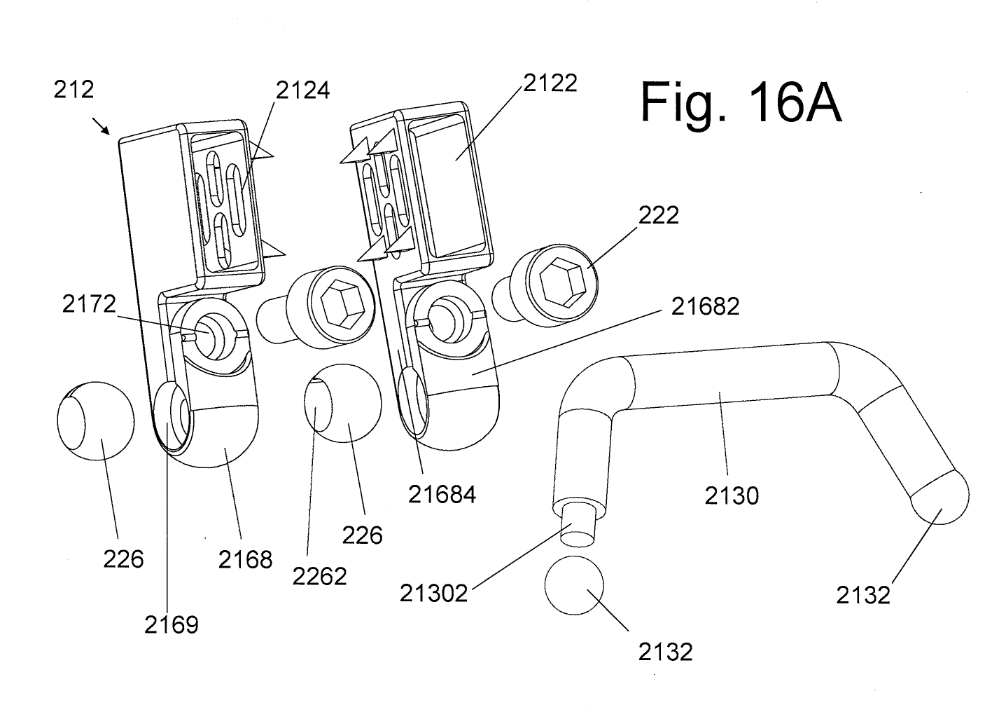

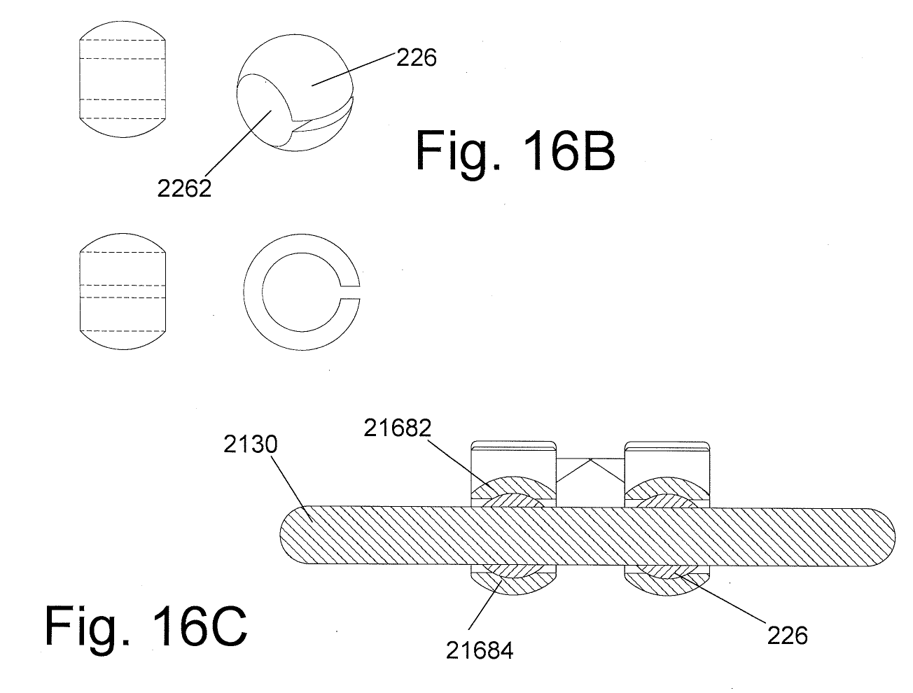

An additional device embodiment is shown in FIG. 15 and a disassembled view is shown in FIG. 16. Device 22 has two members 212. Each member 212 has compartment 2122 that is adapted to receive and house a bone graft or bone graft substitute. Multiple bores 2124 are contained within the medial wall of compartment 2122. Bores 2124 permit communication between the bone graft material within compartment 2122 and the adjacent spinal bone, so that a bony fusion could be established between the bone graft within compartment 2122 and the adjacent spine. The compartment 2122 has an upper opening, a lower opening and the possibility of multiple medial wall opening. The upper and lower opening permit placement of the bone graft material through the upper opening and communication between the contents of compartment 2122 and the lamina of the bone to which the device is attached. The medial wall openings permit communication between the contents of compartment 2122 and the boney surface of the spinous process to which it is attached. Multiple spiked protrusions 2126 protrude from the medial wall of each member 212 and permit device fixation to bone.

Split member 2168 has an upper arm 21682 and lower arm 21684 around central bore 2169. In the assembled state, sphere 226 resides within central bore 2169 of split member 2168. Bar 2130 resides within the central bore 2262 of spherical member 226. Member 226 has a central bore 2262 a side channel so that the spherical member is split on one side. Spherical member 226 is shown in perspective and orthogonal views in FIG. 16B. Threaded locking screw 222 (threads not shown) is adapted to threadedly interact with threaded bore 2172. With advancement of locking screw 222 within threaded bore 2172, the upper arm 21682 and lower arm 21684 of member 2168 are forced towards one another, producing closure of split segment 2168 and reduction of the diameter of central bore 2169. In this way, the split locking sphere 226 is compressed and bar 2130 is immobilized relative to member 212. Thus, with the advancement of screw 222, member 212 is rigidly immobilized relative to bar 2130. A cross-sectional view through the rod and sphere 226 is shown in FIG. 16C.

Bar 2130 has an end protrusion 2132 on each end, wherein the protrusions are preferably spherical. At least one end 2132 is removable so that bar 2130 can be passed through bore 2262 of each locking sphere 226 during device assembly. The removable protrusion 2132 contains a threaded bore that can be threadably attached to threaded end 21302 (threads not shown) after device assembly. In this way, the device is retained in the assembled configuration. Note that the compartment 2122 may contain bores that open onto the side bone, as depicted. As an alternative (or in addition) to the side bores, compartment 2122 may contain at least one bore on the surface that abuts, or is closest to, the lamina portion of the vertebral level to which the device is attached. The latter bore holes would permit bone growth between the fusion material inside compartment 2122 and the lamina that is adjacent (and anterior) to the device.

The implantation procedure for device 22 is similar to that of device 105. If desired, decompression is performed by the surgeon as previously described. Each member 212 is placed on opposing sides of the spinous process of the superior vertebral bone. Bar 2130 is then rotated and positioned until each end protrusion 2132 abuts the lamina surface of the inferior vertebral bone. A compression device (not shown) is used to forcibly compress and drive members 212 towards one another. Spiked protrusions 2162 are forcefully driven into each side of the spinous process of the superior vertebral bone. Each set screw 222 is then advanced so as to lock members each member 212 to bar 2130 and immobilize all members of device 22. Cavity 2122 is then packed with bone graft material through the upper opening. The bone graft material may be forced through the lower opening and onto the lamina below. The bone graft material may also make contact with the bony side surface of the spinous process to which device 22 is attached.

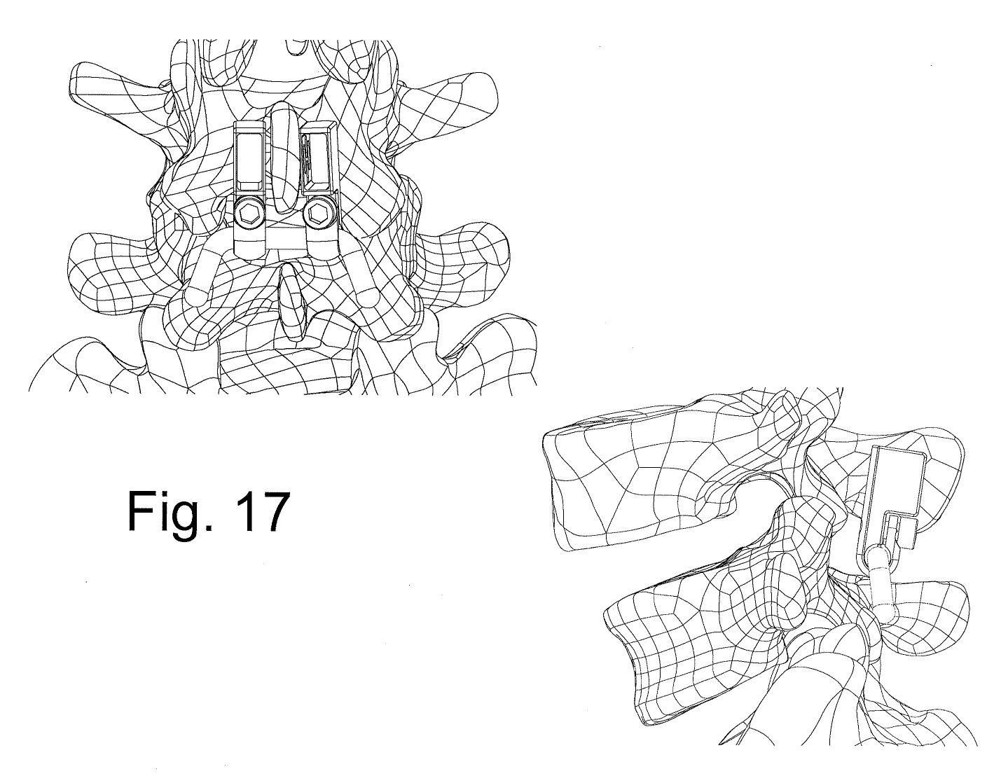

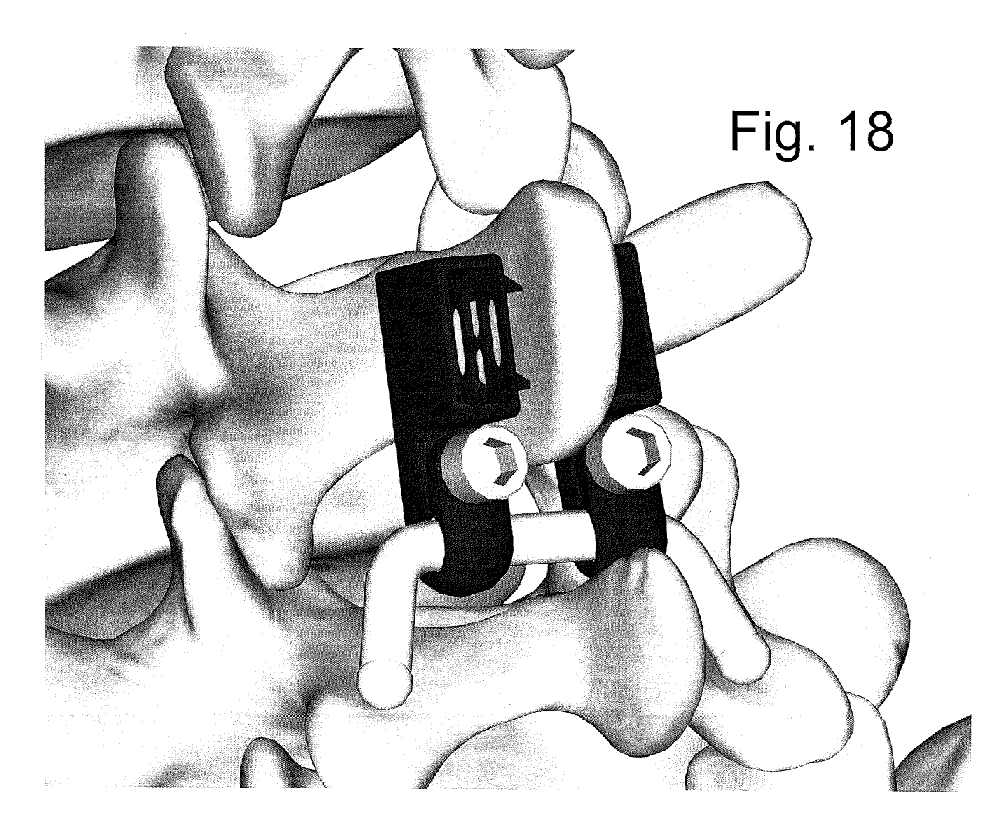

The device is shown attached to the spine model in FIGS. 17 and 18. Again, those skilled in the art will appreciate that actual vertebral bodies include anatomical details not shown in these figures. With device implantation, bearing surfaces 2132 prevent the anterior movement of the superior vertebral bone relative to the inferior vertebra bone in the horizontal plane and prevent the formation or exacerbation of an anterior spondylolisthesis between the two vertebrae. Further, since the inferior lamina of the inferior vertebral bone is angled so that the superior edge is anterior to the inferior edge, the bearing surface 3222 will also limit vertebral extension between the superior and inferior vertebral bones.

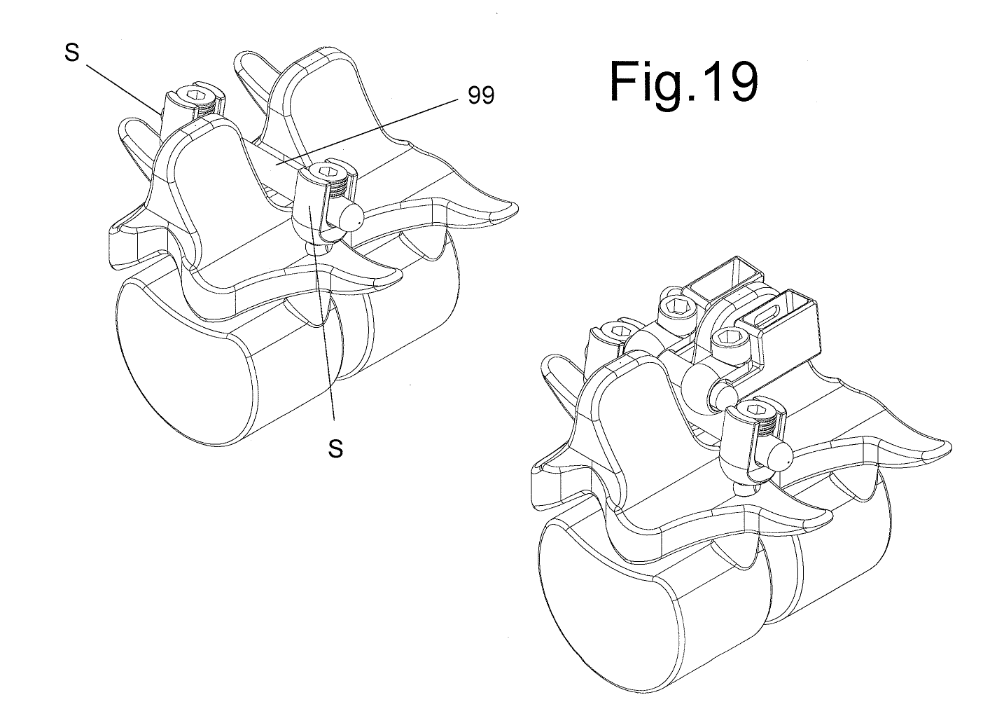

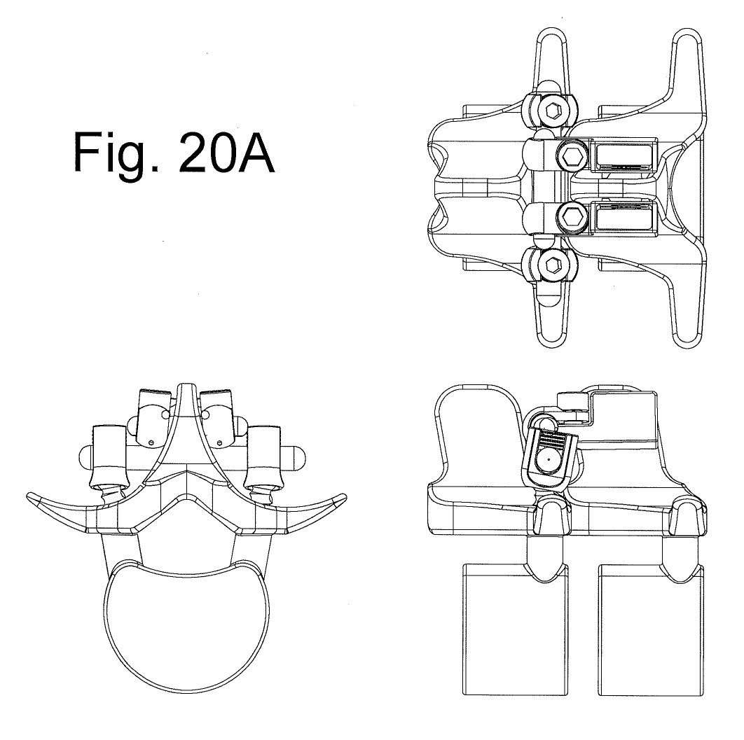

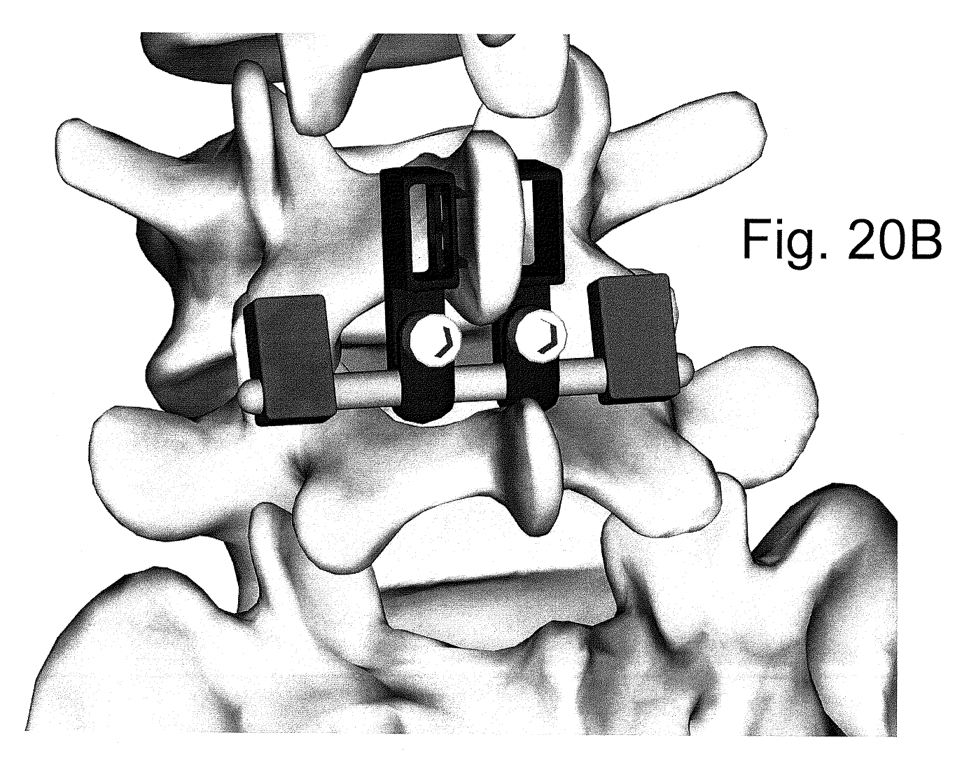

FIG. 19 illustrates a different method of use for device 22. Once again, those skilled in the art will appreciate that actual vertebral bodies include anatomical details not shown in FIG. 19. In this method, bone screws S are placed into the pedicle portion of the inferior vertebral bone and a bar 99 is used to connect each of the bone screws—as shown in FIG. 19. Device 22 has members 212 connected with a short, straight rod. Device 22 is attached to the spinous process of the superior vertebra and an inferior surface of the device is placed in contact with the bar 99, that connects screws S. In this way, members 212 and the connecting bar 99 form the abutment surface that resist vertebral movement in the horizontal plane. Orthogonal views are shown in FIG. 20A.

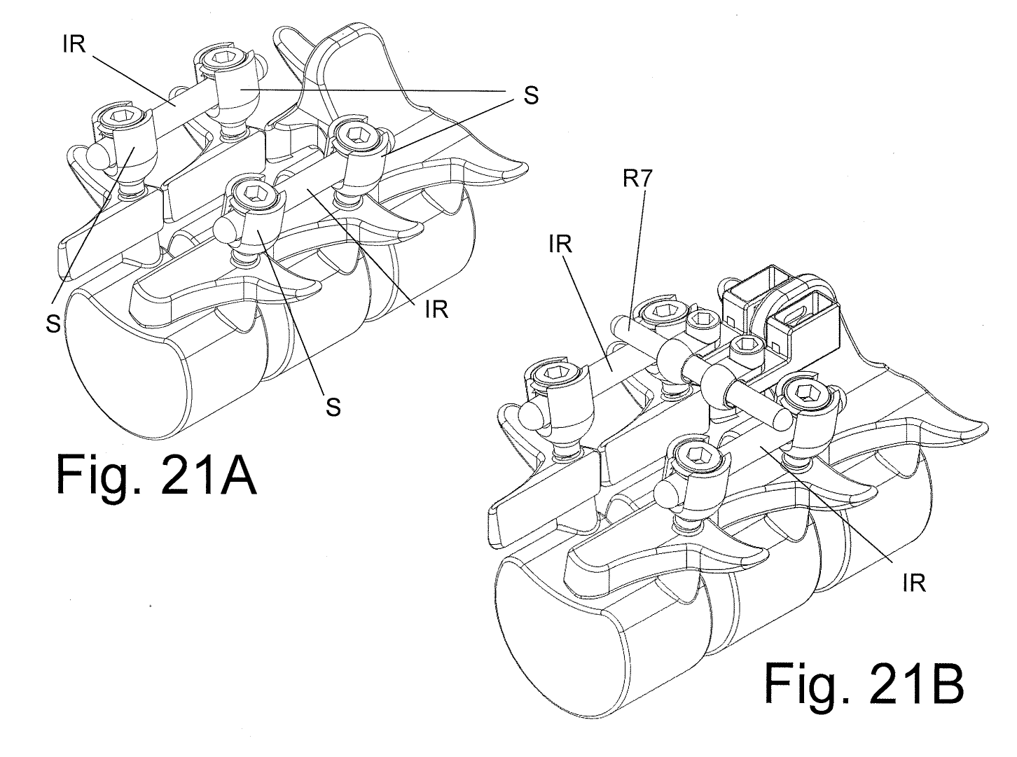

An additional method of use is contemplated and illustrated in FIGS. 21A and 21B, wherein the straight rod that connects each member 212 is shown in FIGS. 19 and 20 is used to span the distance between each of two rods that have been used to interconnect bone screws, wherein the bone screws and interconnecting rod have been placed at each of the two vertebral bones that are inferior to the vertebral bone onto which device 22 is rigidly attached. FIG. 21B illustrates the implanted device 22. This method of device use is particularly applicable in patients who have a fusion, whether at a current or prior operation, of two vertebral bone that are inferior to the implantation site of device 22. Decompression may be undertaken as shown in FIG. 7 and the spinous process of the vertebral bone immediately above the fusion is preserved. Device 22 is attached to the spinous process of the superior vertebral bone and the interconnecting rod of device 22 is positioned to abut the interconnecting rod IR that couple the bone screws S.

If the fusion of the two inferior bones was performed at a prior operation, then the fusion mass placed around interconnecting rods IR may have grown to completely surround and encase each interconnecting rod IR. Should that occur, the interconnecting rod (R7) of device 22 may be positioned to abut directly the bone of the fusion mass that surrounds rod IR at the time of device 22 implantation.

An additional method of use is contemplated (not shown), wherein device 22 is attached to the superior vertebral bone as shown in FIG. 21B. Each end of the straight rod that connects each member 212 is then positioned immediately posterior to the posterior surface of a superior articulating process (SAP) of the inferior vertebral bone (that is, the end of the rod is positioned to abut the segment of lines B of FIGS. 3c and 3D). In the current method, prior screws S and rod IR are not present. In this way, anterior migration of the superior vertebral bone relative to the inferior vertebral bone is prevented by the abutment of the interconnecting rod of device 22 and the posterior surface of the SAP of the inferior vertebral bone. See FIGS. 3C and 3d for the areas of contact along the SAP of the inferior vertebral bone. It should be noted that the rod may have flattened plate-like abutment surfaces at each end that is adapted to abut the SAP—as shown in FIG. 20B.

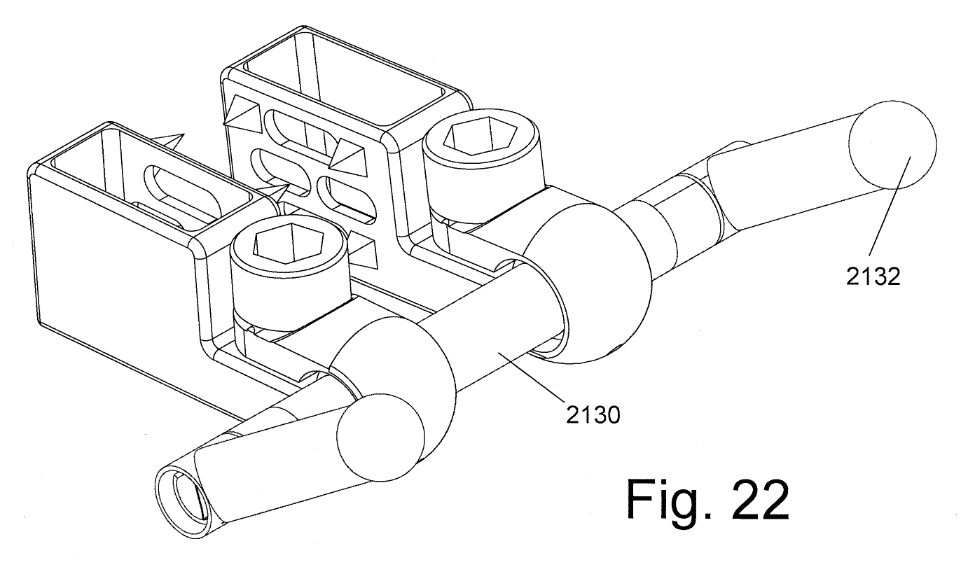

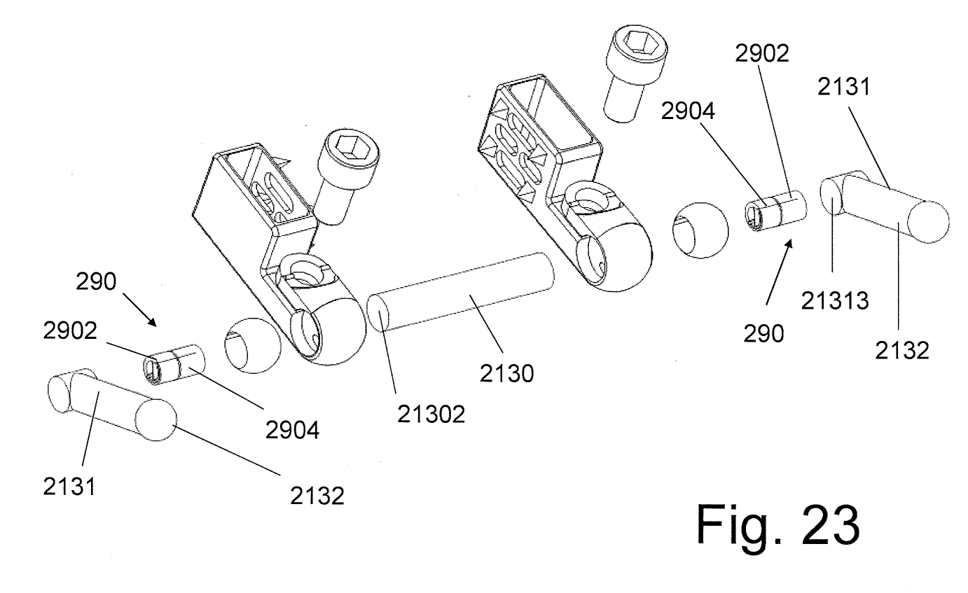

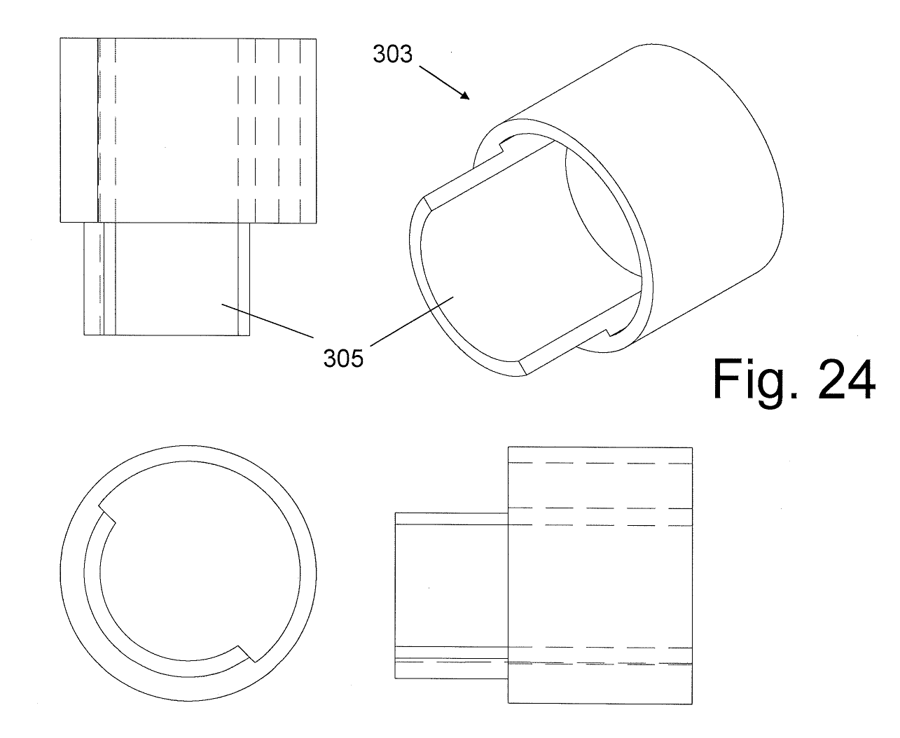

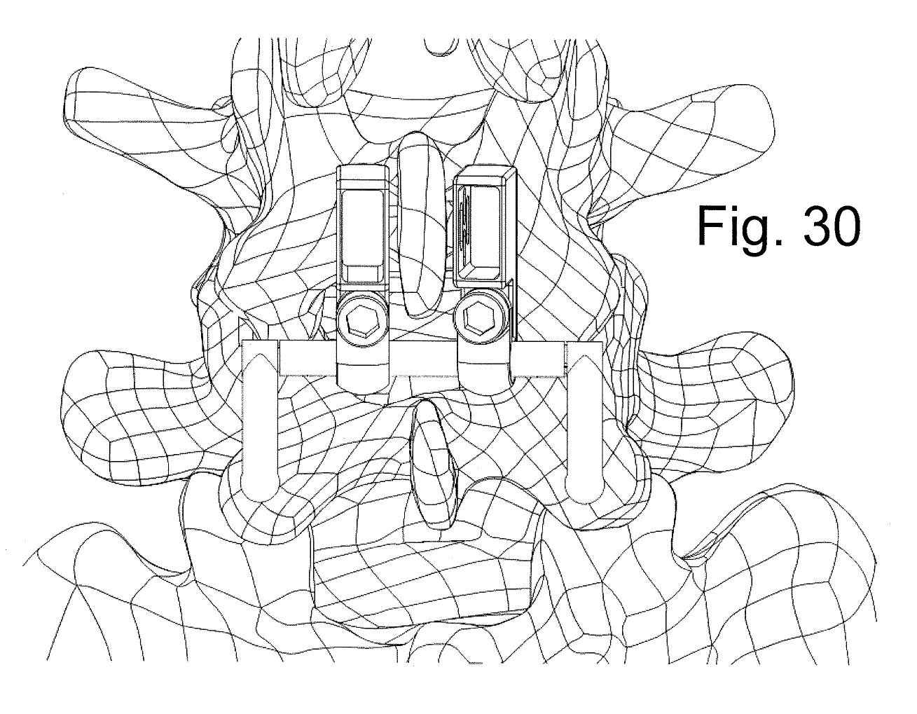

FIGS. 22 and 23 illustrate an additional embodiment. While similar to the preceding device, this embodiment permits each of protrusion 2132 to rotate relative to bar 2130 through the action of pivot member 290. The assembled device is shown in FIG. 22 while the exploded view is shown in FIG. 23. The implanted device is shown in FIG. 30.

In this embodiment, members 212 are similar to those of device 22. The interconnecting rod member differs in that the rod had center component 2130 and two side components 2131. Center component 2130 has an opening 21302 on each end that is adapted to accept end 2904 of pivot member 290. Each side component 2131 has a first spherical end 2132 and a second end that contains an opening 21313, wherein opening 21313 is adapted to accept end 2902 of pivot member 290. When in the assembled state (FIG. 22), each side component 2131 can pivot relative to component 2130 about the long axis of component 2130. The pivot member is biased to return each component 2131 to a neutral position relative to component 2130 after a deflecting force acting upon the device has dissipated.

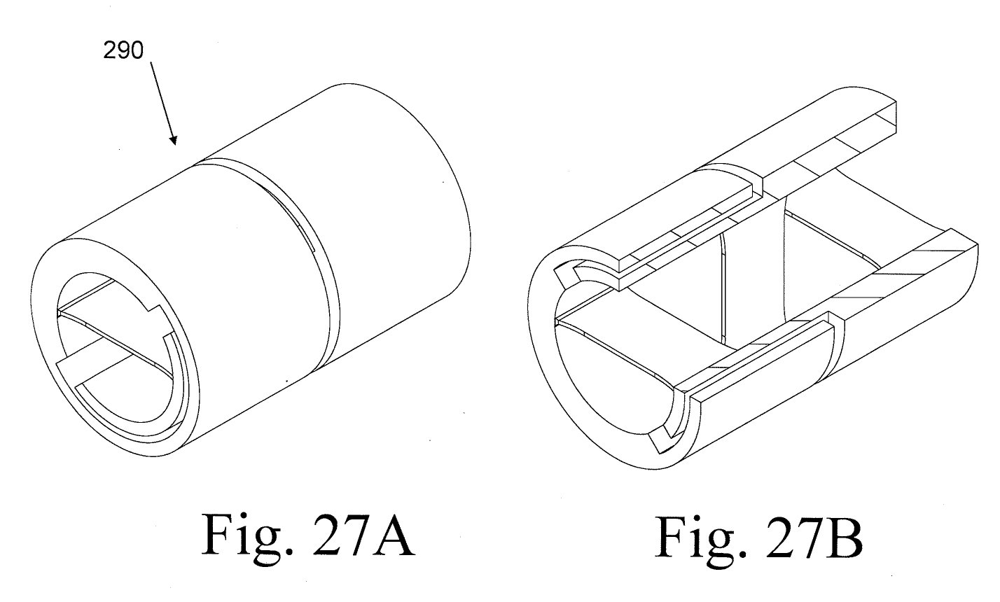

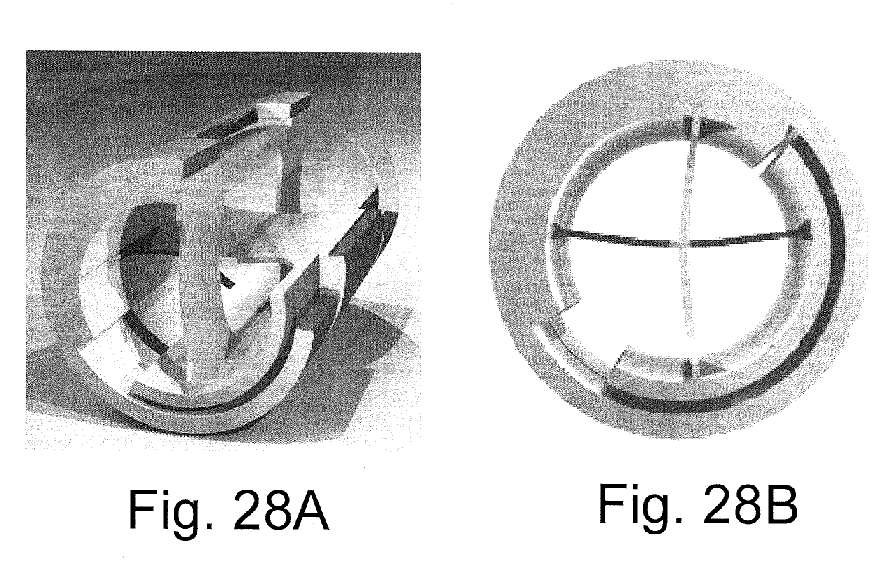

FIGS. 27A and 27B show the assembled pivot member 290 and a partial section view of member 290, respectively. The pivot member 290 is formed by a plurality of sections. Member 290 is a flexure based bearing, utilizing internal flat crossed slats 307, encapsulated in a cylindrical housings 303, to provide precise rotation with low hysteresis and little frictional losses. The bearing is relatively friction-free, requires no lubrication, and is self-returning. Member 290 can resist rotational movement away from a neutral state and the extent of resistance to rotation is directly related to the extent of rotation. Member 290 has high axial stiffness.

The pivot member is a flexible rotational articulation that contains a first hollow cylindrical member 303 which is comprised of an outer surface with a defined radius from a longitudinal central axis, an inner surface with a defined radius from a longitudinal central axis, a defined thickness and defined internal cavity that is contained within the inner surface. With reference to FIG. 24, at least one cylindrical tab 305 extends from one end of said member wherein the tab circumferentially extends less than one hundred and eighty degrees around its longitudinal central axis.

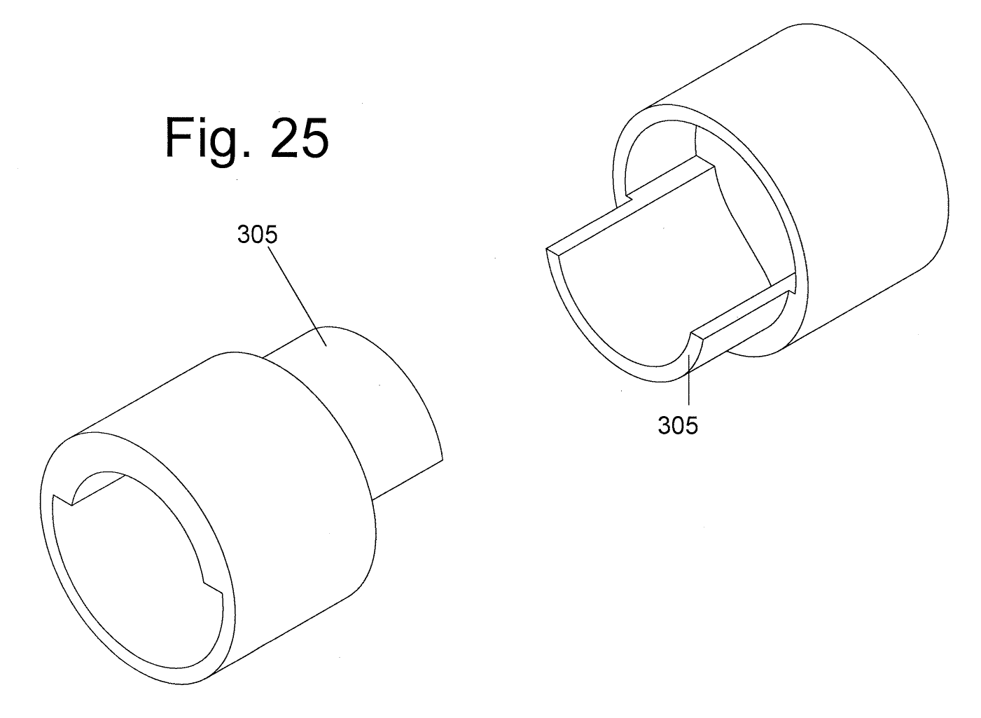

With reference to FIG. 25, a second hollow cylindrical member is comprised of an outer surface with a defined radius from a longitudinal central axis, an inner surface with a defined radius from a longitudinal central axis, a defined thickness and defined internal cavity that is contained within the inner surface. At least one cylindrical tab extends from one end of said member wherein the tab circumferentially extends less than one hundred and eighty degrees around its longitudinal central axis. Preferably, but not necessarily, the first and second cylindrical members may be identical.

With reference to FIG. 25, the first and second members are axially aligned, wherein one tab of the first member is positioned within the internal cavity of the second member and one tab of the second member is positioned within the internal cavity of the first member.

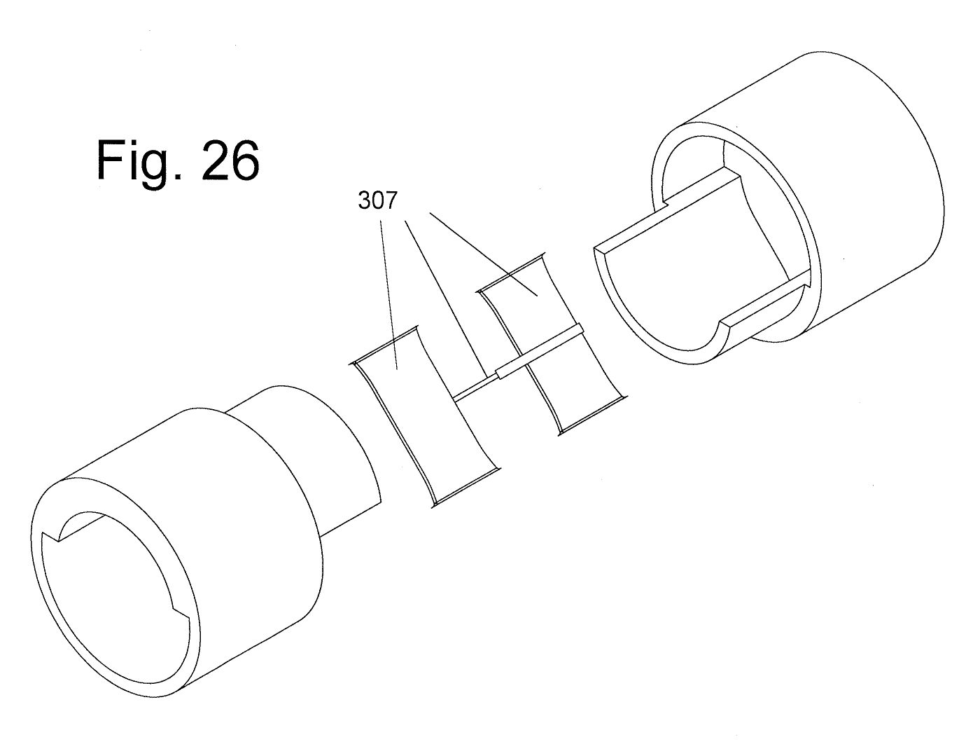

At least one flexible element 307 connects the inner surface of the tab member 305 of the first member 303 with the inner surface of the second member 303 and at least one flexible element connects the inner surface of the tab member 305 of the second member with the inner surface of the first member 303 so that said first and second members 303 may smoothly rotate relative to one another about a central longitudinal axis. The elements 307 are joined with the inner surfaces of members 303 and tabs 305 using any method that is known in the art to join these members—including welding and the like. An exploded view is shown in FIG. 26. An assembled view is shown in FIG. 27A and a partial section view is shown in FIG. 27B.

The device is commercially available from the Riverhawk company of New Hartford, N.Y. 13413. The web site http://www.flexpivots.com describes the device in detail and the totality of the information contained within the web site is hereby incorporated by reference in its entirety. Further, prior disclosures of similar flexible pivot devices have been made in U.S. Pat. Nos. 5,620,169, 6,146,044 and 6,666,612. The disclosure of each of these patents is hereby incorporated by reference in its entirety.

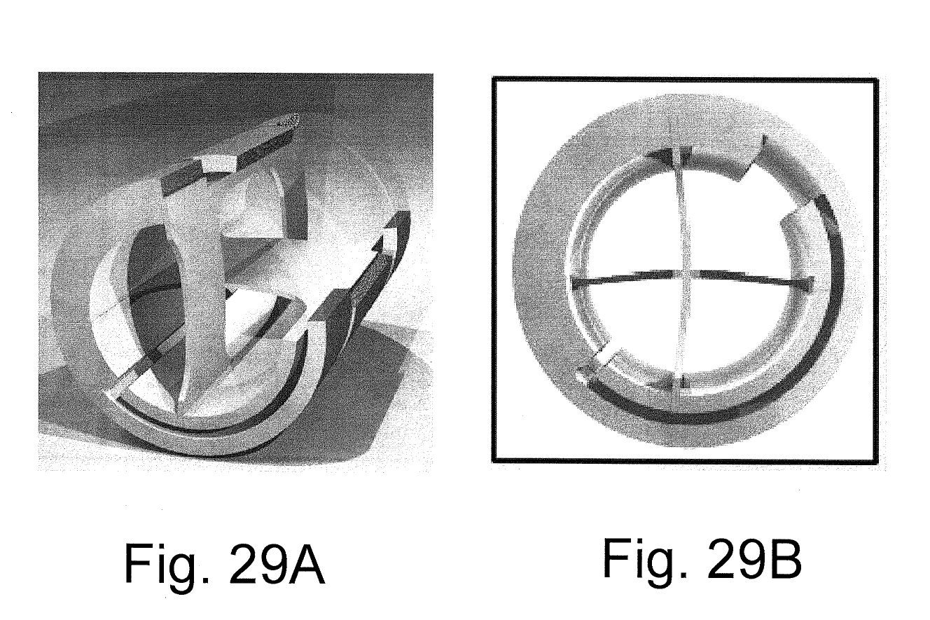

Pivot member 290 is housed within a cavity on the end of each arm 2131 and bar 2130. Each of the two cylindrical housing members of the pivot member is rigidly attached to end cavity of either arm 2131 or bar 2130 so that rotation of arm 2131 about the long axis of bar 2130 produces deformation of the internal flat crossed slats of member 290. FIGS. 28 and 29 illustrate the deformation of the internal flat crossed slats with movement of the member to either rotational extreme, wherein FIGS. 28A, 28B, 29A, and 29B show the pivot member 290 alone and not the pivot member and the device of FIG. 22. In this way, member 290 functions to return each component 2131 to a neutral position relative to competent 2130 after a deflecting force that has been acting upon the device has dissipated.

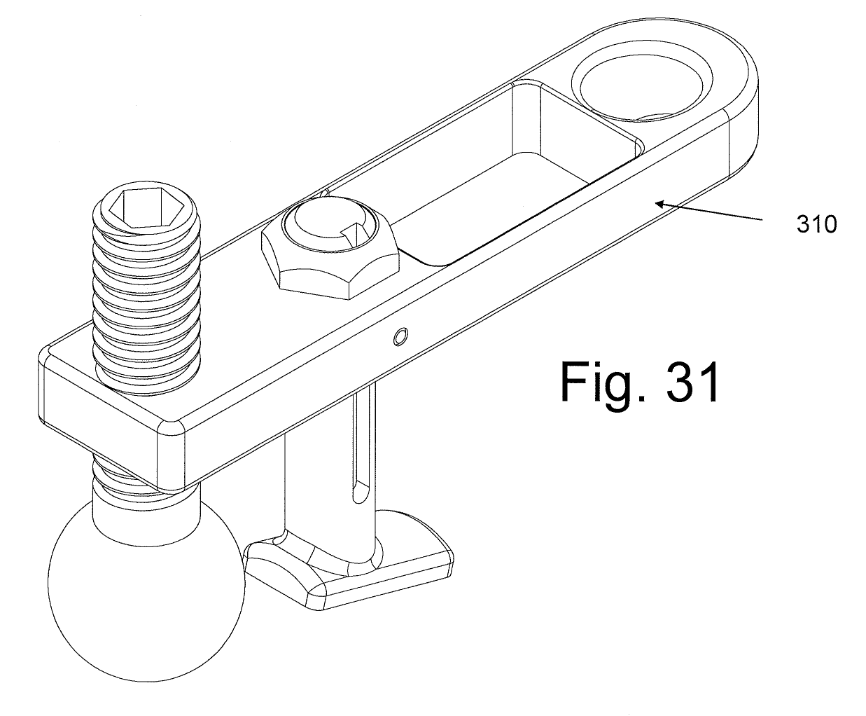

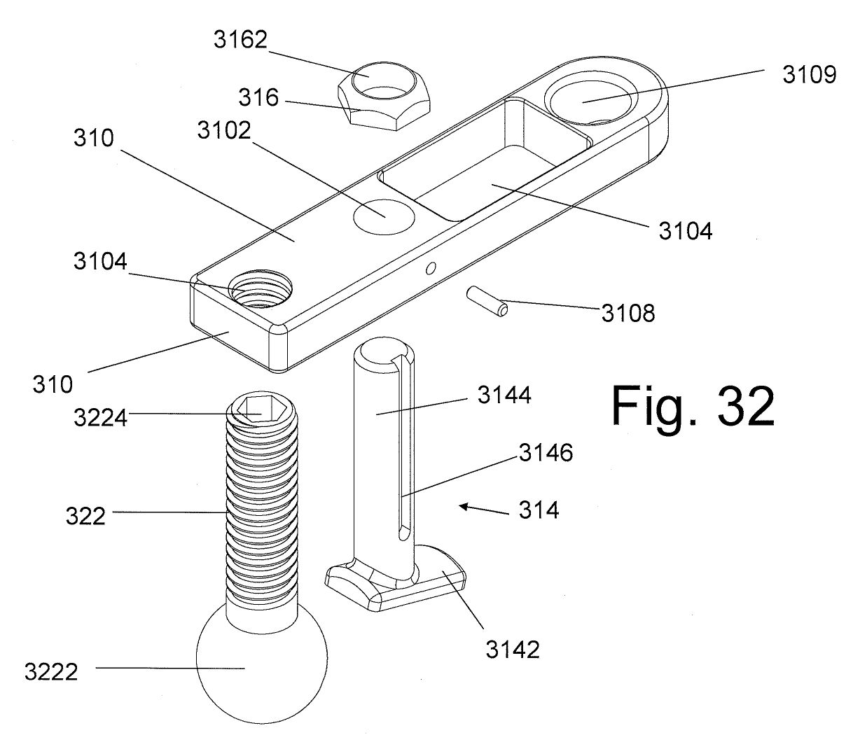

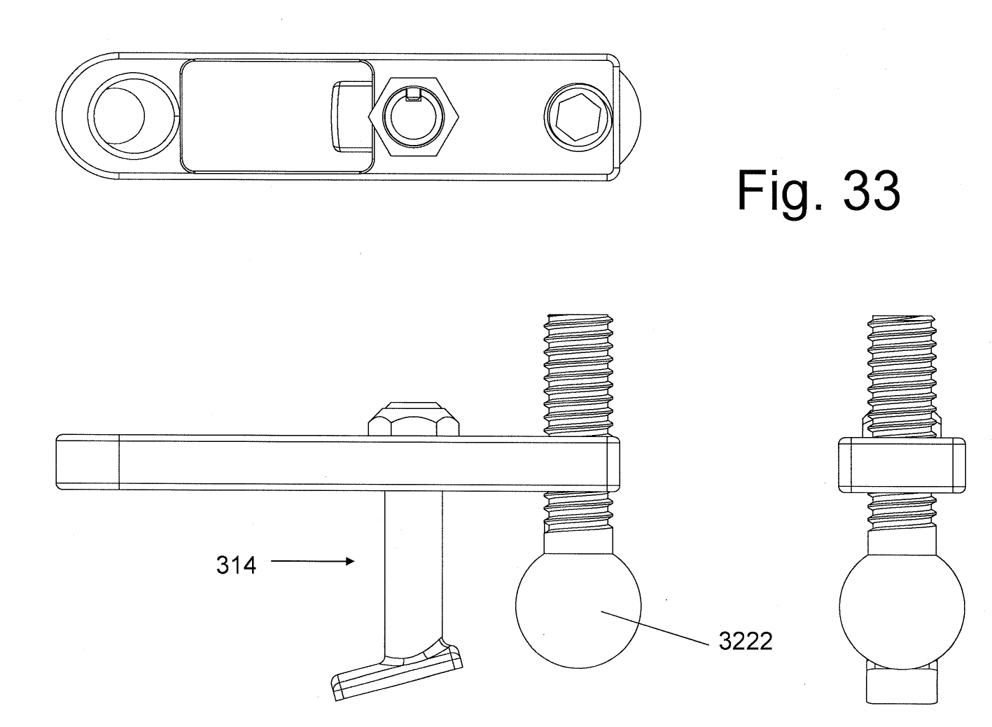

An additional embodiment is shown in FIGS. 31 to 35. A oblique view of the assembled device is shown in FIG. 31 while an exploded device is illustrated in FIG. 32. The device is shown in multiple orthogonal planes in FIG. 33. The device is comprised of member 310, hook member 314, locking nut 316 and member 322 with end bearing surface 3222. In an embodiment, member 310 contains a cavity 3104 that is adapted to contain a bone graft or bone graft substitute. When implanted onto the spine, the material contained within cavity 3104 communicates with the adjacent bone through a opening at the bottom of the cavity and forms a fusion mass between the contents of cavity 3104 and the adjacent bone. The side walls of cavity 3104 may be angled so that the cavity opening that abuts the bone is smaller than the cavity opening of the top surface of member 310. In this way, the fusion mass will resist movement of the device away from the bone to which the device is attached and fused.

Hook member 314 has foot segment 3142 that is adapted to anchor onto an undersurface of a bone segment to which the device is attached. While not shown, cylindrical post segment 3144 of member 314 is threaded (threads not shown). Segment 3144 also contains side channel 3146 and rests within non-threaded bore 3102 of plate 310. Locking nut 316 portion has treaded bore 3162 (threads not shown) that is adapted to accept and threadedly cooperated with threaded cylindrical segment 3144 of member 314. At device assembly, pin 3108 is pressed into a side bore of member 310 and into channel 3146 of segment 3144. The pin prevents rotation of hook member 314 relative to member 310, during, for example, tightening/loosening of locking nut 316. Member 322 has threaded cylindrical member that rests within threaded bore 3104 of member 310. One end of member 322 contains a depression 3224 (or protrusion) adapted to accept a screw driver that is adapted to engage and rotate member 322. At a second end, member 322 contains a bearing surface 3222.

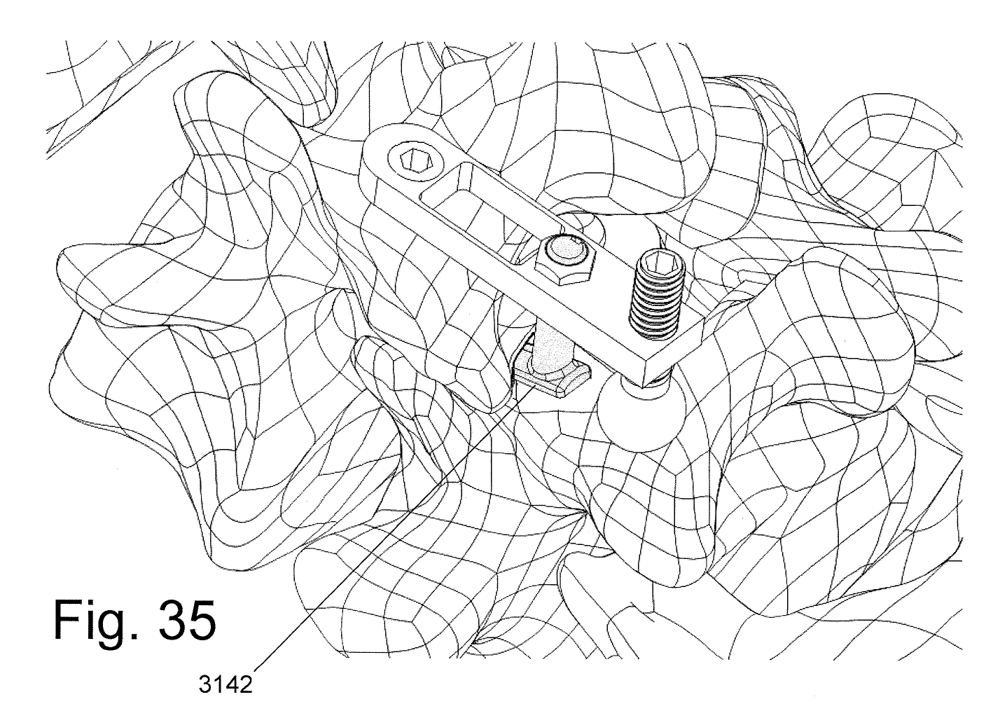

The device is adapted to attach onto a portion of the upper vertebral bone and form an abutment surface with the lamina and posterior aspect of the IAP of the lower vertebral bone. At a first end, the device is attached to the upper vertebra by a fastener, such as a bone screw, that rests within bore hole 3109. The bore is preferably conical, spherical or otherwise adapted to permit movement of the fastener head in one or more planes and the fastener is preferably affixed to the pedicle portion of the superior vertebral bone. Hook member 314 is used to attach a second end of member 310 onto a portion of the lamina or a segment of IAP of the superior vertebral bone. The foot segment 3142 is adapted to capture the undersurface (anterior surface) of the lamina and/or IAP of the superior vertebral bone—as shown in FIG. 35. The foot segment is shaded to contrast with the surrounding bone. Note that the foot segment captures the edge of the lamina (or a cut or uncut edge of the IAP) of the superior vertebral bone between the foot segment and member 310.

After the device is affixed to the upper vertebral bone, member 322 is actuated until bearing surface 3222 abuts the lamina or posterior aspect of the IAP of the inferior vertebra. The bone adjacent to (anterior to) cavity 3104 is decorticated in order to promote bone fusion and a bone forming material is packed into cavity 3104. With time, the material of cavity 3104 will fuse with the underlying bone segment (lamina) of the superior vertebra bone and provide an additional attachment point for the device. Preferably, a device is implanted on each side of the vertebral midline (so as to implant two devices per functional spinal unit). In use, bearing surface 3222 prevents the anterior movement of the superior vertebral bone relative to the inferior vertebra bone in the horizontal plane and prevents the formation or exacerbation of an anterior spondylolisthesis between the superior and inferior bone. Further, since the inferior lamina of the inferior vertebral bone is angled so that the superior edge is anterior to the inferior edge, the bearing surface 3222 will also limit vertebral extension between the superior and inferior vertebral bones.

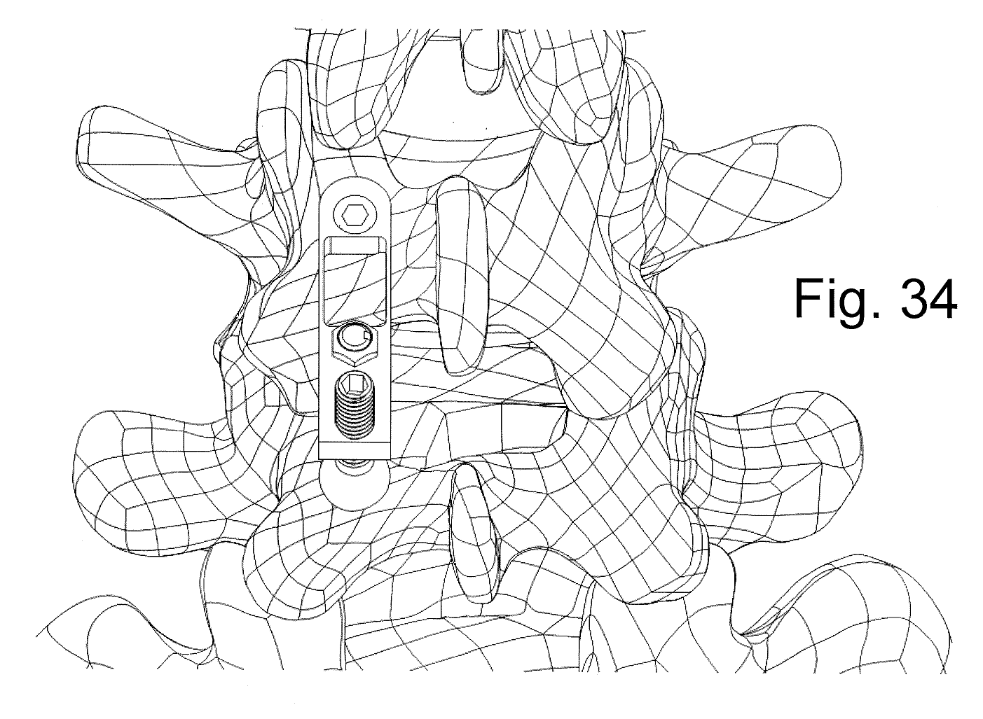

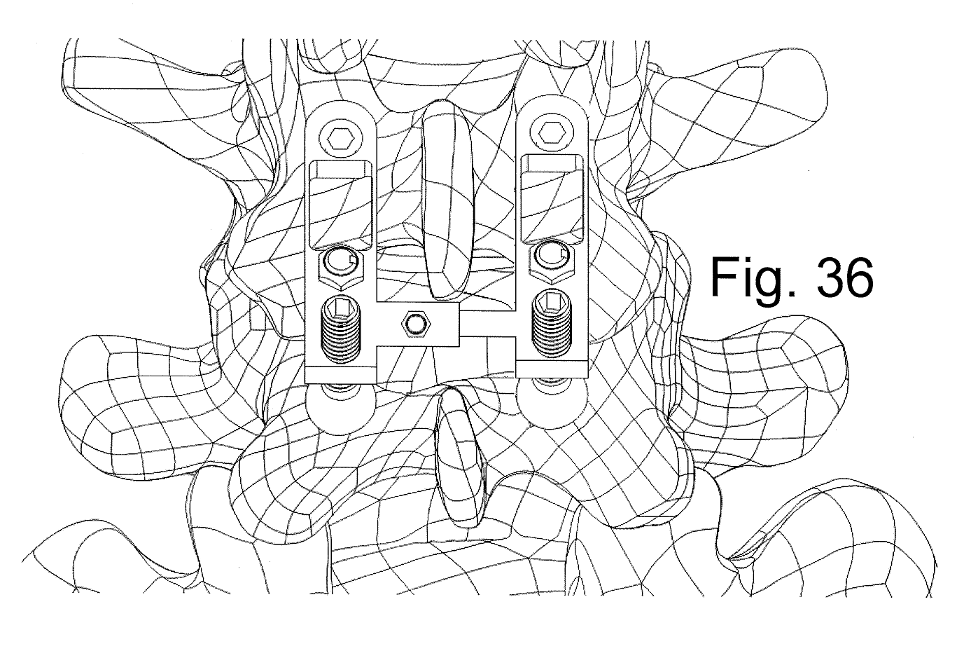

FIG. 36 illustrates an embodiment wherein two of the devices shown in FIG. 34 are connected across the midline. Preferably, the connection segment permits the relative movement of the two devices so that the total width of the device can be adjusted. While not illustrated, the connection segment may be made of sufficient length in order to span from the inferior aspect of the spinous process of the superior vertebral bone to the superior surface of the spinous process of the inferior vertebral bone.

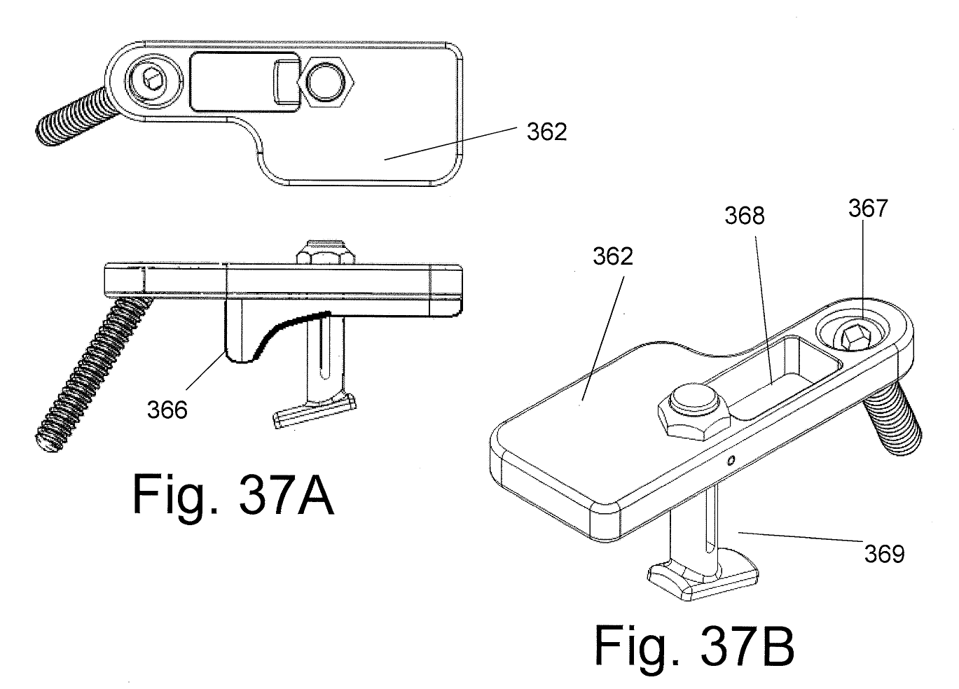

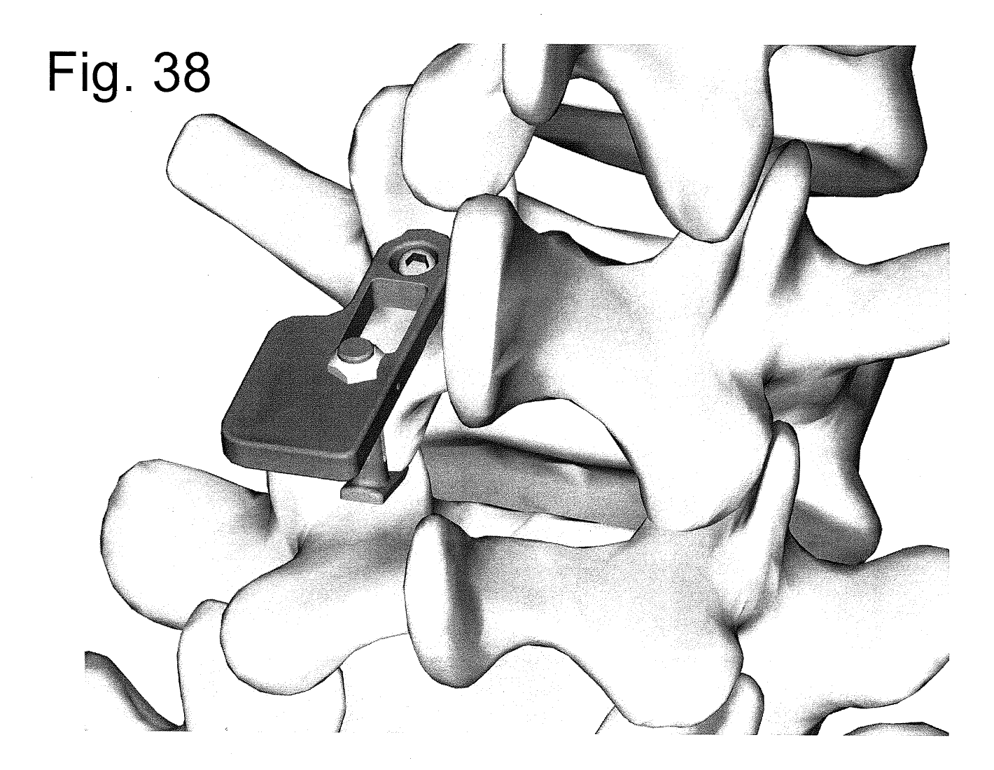

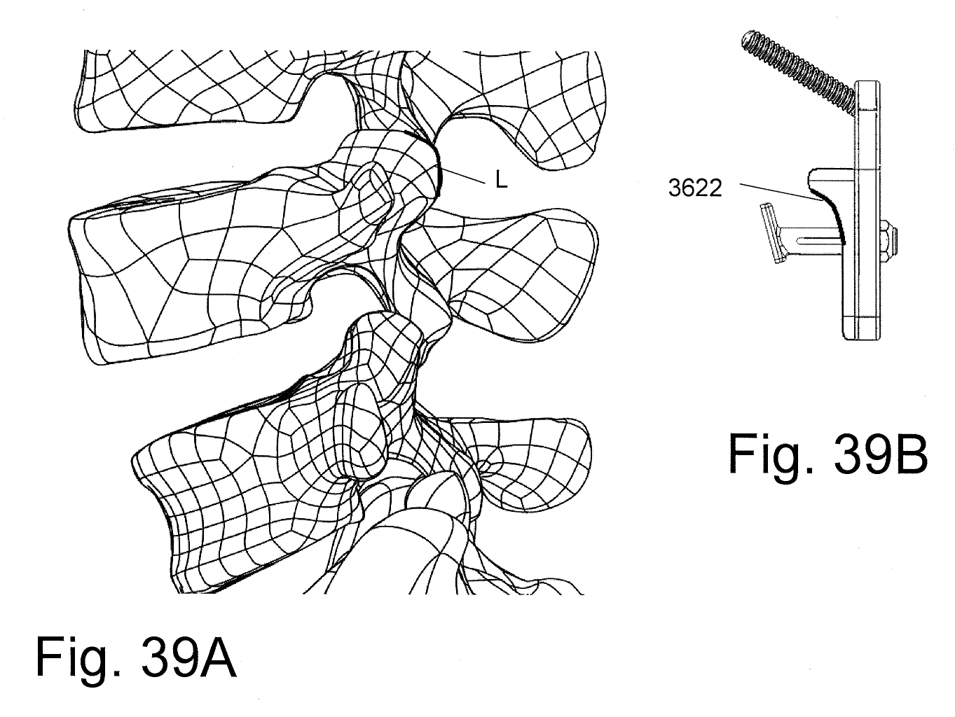

FIGS. 37A and 37BB show multiple views of an additional embodiment. While similar to the embodiment of FIG. 31, the current embodiment has a lateral extension 362 that forms an abutment surface with the posterior surface of the superior articulating process (SAP) of the inferior vertebral bone. Further, the device preferably, but not necessarily, contains an extension 366 from the inferior surface which can abut the superior aspect of the SAP of the inferior vertebral bone. As before, the device anchors onto the superior vertebral bone using a bone fastener 367 at one end, a bone hook 369 at a second end and a bone fusion cavity 368 there between. The device is shown anchored to the spine in FIG. 38. FIG. 39A shows the lateral aspect of the spine. When implanted, the surface 3622 of the inferior surface of the device abuts the surface “L” of the superior articulating process (SAP) f the lower vertebral bone. In use, bearing surface 3622 prevents the anterior movement of the superior vertebral bone relative to the inferior vertebra bone in the horizontal plane and prevents the formation or exacerbation of an anterior spondylolisthesis between the superior and inferior bone. Further, since extension 366 from the inferior surface of the device abuts the superior aspect of the SAP of the inferior vertebral bone, the bearing surface 3622 will also limit vertebral extension between the superior and inferior vertebral bones.

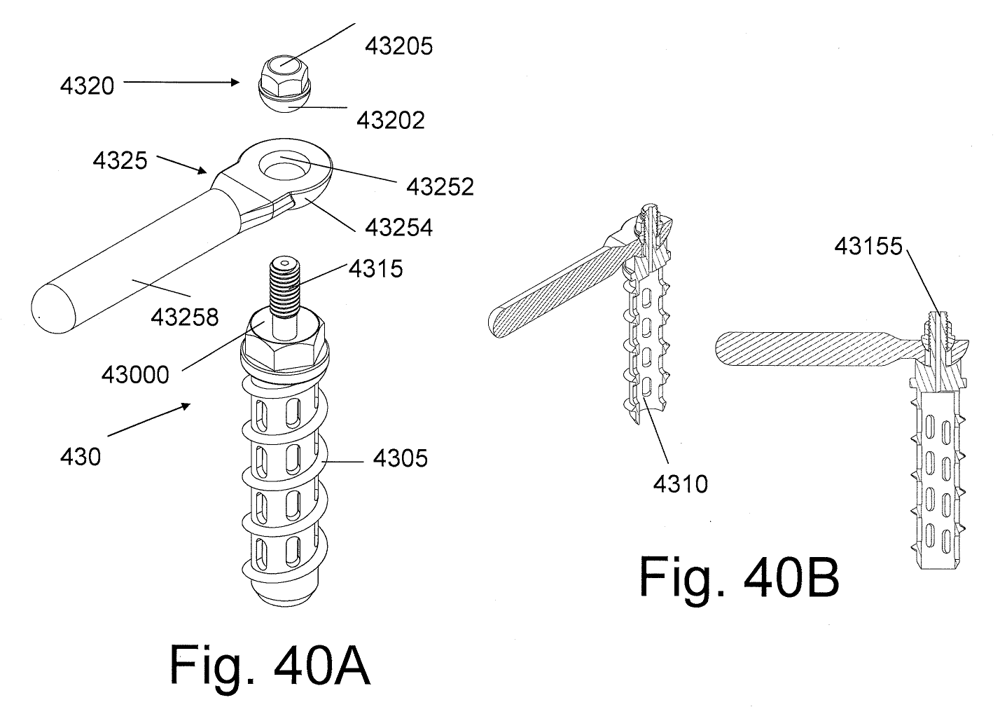

An additional embodiment is shown in FIG. 40A (exploded view) and FIG. 40B (sectional views). Member 430 comprises a body that extends along a longitudinal axis. (While depicted as cylindrical, the body may be alternatively configured to be conical, a frustum, acorn-shaped, or any other appropriate geometric configuration.) A raised helical thread 4305 winds around the outer surface of the body. As shown in the cross-sectional views of FIG. 40B, the body includes an internal chamber 4310 defines by a cylindrical outer wall. A plurality of openings extend through the cylindrical outer wall. The openings permit communication between a bone graft material that has been implanted within internal chamber 4310 and the adjacent spinal bone, so that a bony fusion can be established between the bone graft within chamber 4310 and the adjacent spinal bone.

With reference still to FIGS. 40A and 40B, a shank 4315 extends upwardly from the body. The shank 4315 has a threaded outer surface that threadbly mates with a locking nut 4320. Shank 4315 has central bore 43155 adapted to accept guide pins during implantation. Locking nut 4320 has a rounded bottom surface 43202 that mates with a complementary-shaped, rounded seat of a member 4325. Locking nut 4320 also has threaded bore 43205 (threads not shown). The spherical bottom of locking nut 4320 interacts with the complimentary spherical cut out 43252 of member 4325. Spherical bottom 43254 of member 4325 interacts with spherical surface 43000 of member 430. This permits member 4325 to assume a variable spatial orientation relative to member 430 and to be locked into that position by nut 4320. Member 4325 has abutment surface 43258 that is sized and shaped to abut a bony surface.

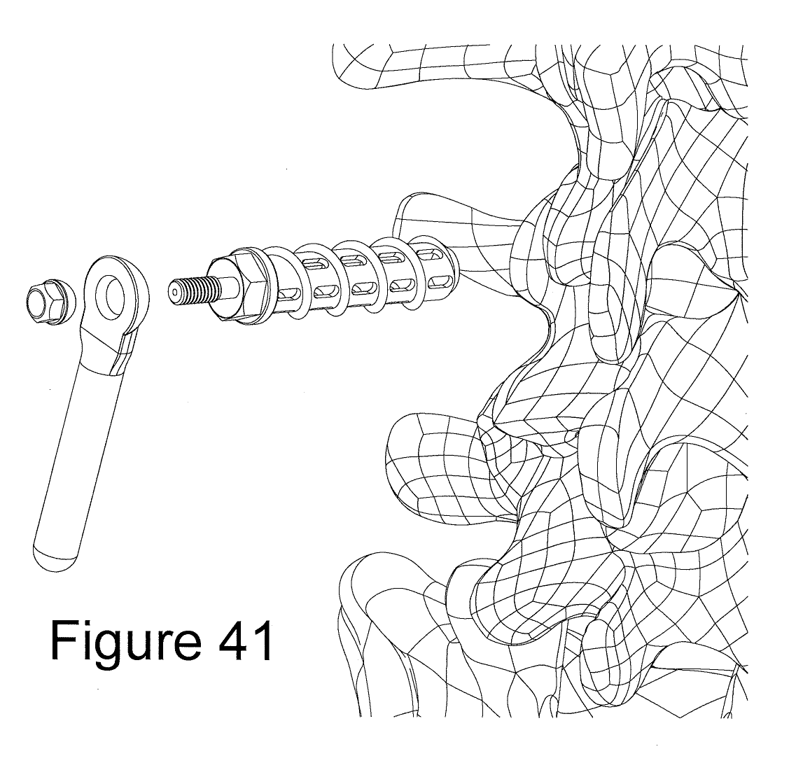

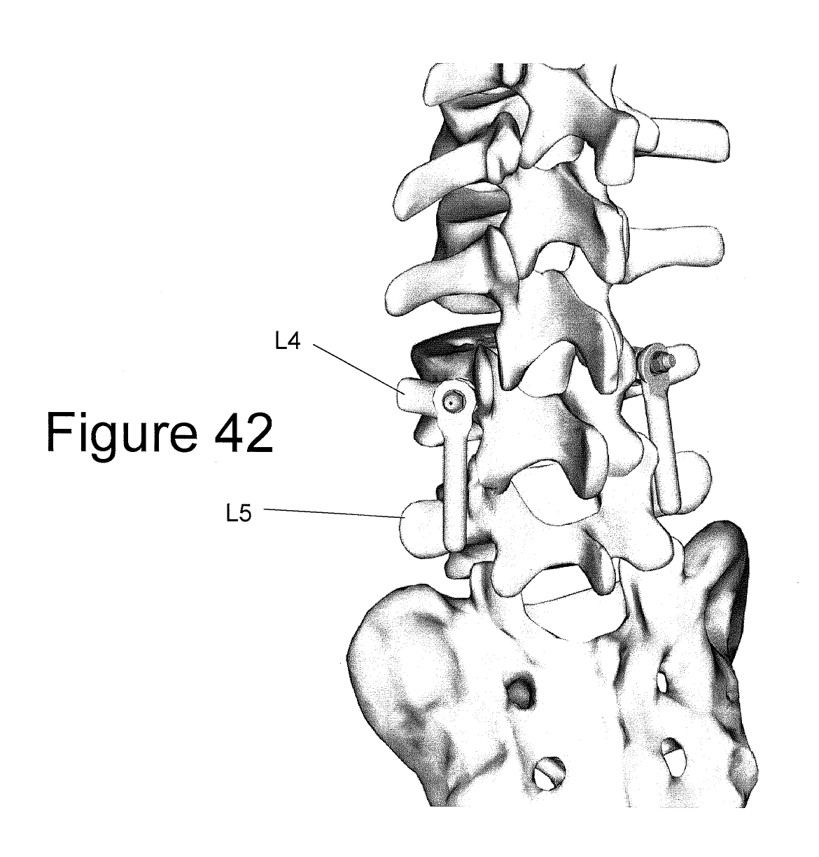

After packing cavity 4310 with bone graft material, device 430 is positioned at or about point 811 (of FIG. 1B) of the superior vertebral bone and then inserted into the underlying pedicle portion of the bone. For clarity of illustration, the procedure is described as occurring on the left side vertebral midline of the superior vertebral bone but may occur on either or both sides of the vertebral bone (FIG. 41). Member 4325 is attached to device 430 so that segment 43254 abuts seat 43000 of device 430. Member 4325 is oriented so that the abutment surface 43258 abuts region 811 of the inferior vertebral bone on the same side of the vertebral midline (the left side as noted above). The locking nut 4320 is locked so that the device is rigidly immobilized. With time, a fusion mass with develop between the bone graft material in cavity 4310 and the adjacent vertebral bone. In this way, the device is rigidly attached to the superior vertebral bone. Bearing surface 43258 prevents the anterior movement of the superior vertebral bone relative to the inferior vertebra bone in the horizontal plane and prevents the formation or exacerbation of an anterior spondylolisthesis between the superior and inferior bone. FIG. 42 illustrates the device implanted on each side of the vertebral midline and preventing anterior spondylolisthesis of L4 relative to L5.

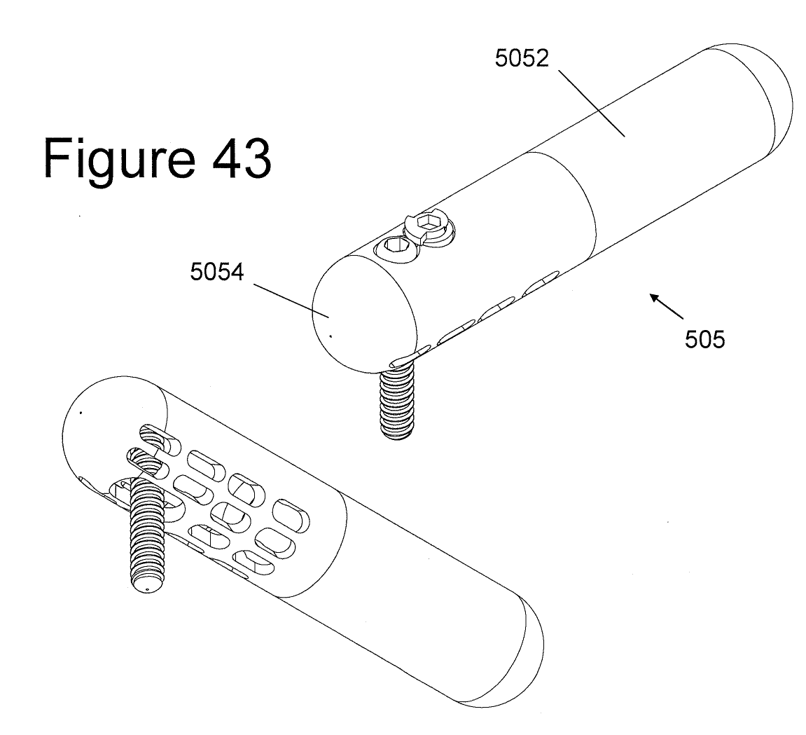

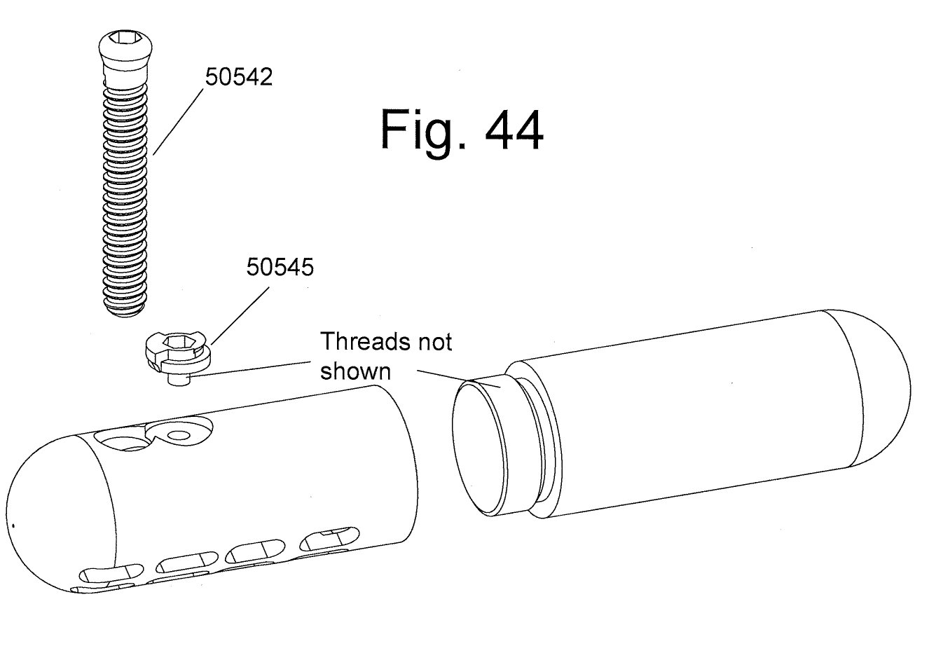

In the preceding embodiment, the device was fused onto the pedicle portion of the superior vertebral bone. In a another embodiment, a solid screw ma be alternatively used to affix the device onto the superior vertebral bone while a hollowed implant that contains an internal cavity that contains bone graft material may be used to fuse onto region 811 of the superior vertebral bone and abut, but not attach onto, region 811 of the inferior vertebral bone. FIGS. 43-45 illustrate an additional device embodiment. Device 505 is comprised of two sections 5052 and 5054 that couple and screw together to form device 505 (threads are not shown).

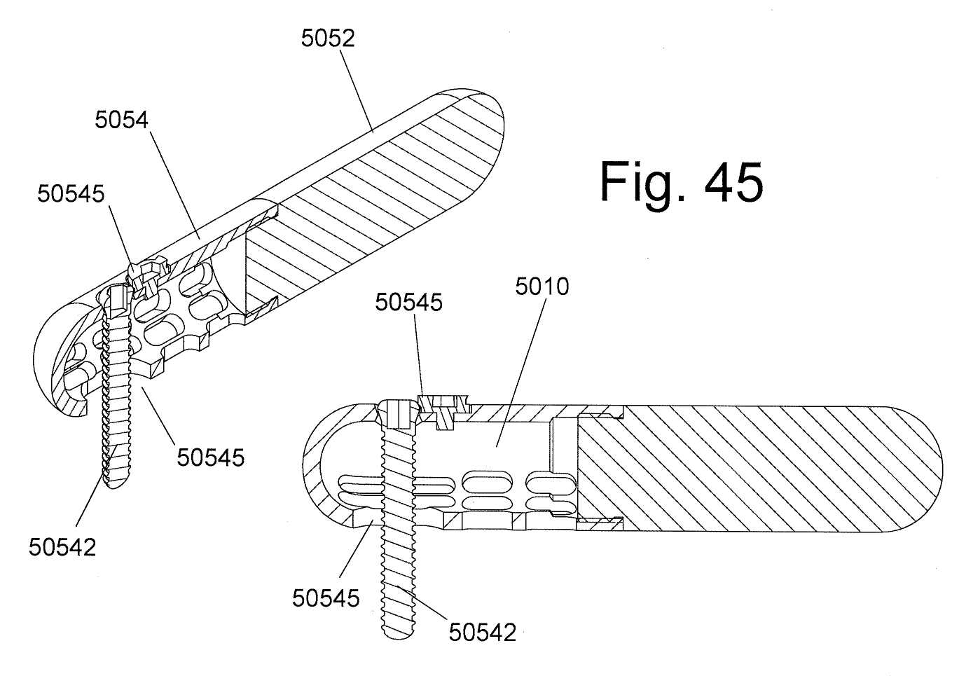

As shown in the cross-sectional views of FIG. 45, member 5054 includes an internal chamber 5010 defines by a cylindrical outer wall. A plurality of openings extend through the cylindrical outer wall. The openings permit communication between a bone graft material that has been implanted within internal chamber 5010 and the adjacent spinal bone, so that a bony fusion can be established between the bone graft within chamber 5010 and the adjacent spinal bone. A bone fastener 50542 is disposed within a bore hole of member 5054. The inferior aspect of the bore hole is slotted to in order to permit fastener 50542 to assume a variable spatial orientation relative to member 5054 and to be locked into that position by locking cam 50545.

At implantation, device 505 is unscrewed into two members 5054 and 5052. Cavity 5010 is packed with bone graft material and the members are reattached to reconstruct the fully assembled device 505. Device is 505 is implanted in the same relative position as the preceding embodiment. Region 810 of the superior vertebral bone is decorticated in preparation for bone fusion. Device 505 is positioned so as to span from region 810 of the superior vertebral bone to region 810 of the inferior vertebral bone (on the same side of the vertebral midline), wherein member 5054 abuts region 810 of the superior vertebra and member 5052 abuts region 810 of the inferior vertebra. Device 505 is rigidly affixed to the superior vertebral bone by placing fastener 50542 through the bore holes of member 5054 that are adapted to accept it and into the pedicle portion of the superior vertebral bone. The locking cam 50545 is actuated in order to lock fastener 50542 to member 5054.

After implantation, device 505 is rigidly attached to the superior vertebral bone. The outer aspect of member 5052 abuts region 810 of the inferior vertebral bone, preventing the anterior movement of the superior vertebral bone relative to the inferior vertebra bone in the horizontal plane and the formation or exacerbation of an anterior spondylolisthesis between the superior and inferior bones.

Each of the embodiments described in preceding disclosure will limit the anterior movement of a superior vertebral bone relative to an inferior vertebra bone in the horizontal plane. While describe as separate embodiments, the various mechanisms may be used in combinations to produce additional assemblies that have not been specifically described herein, but, nevertheless, would fall within the scope of this invention.

The disclosed devices or any of their components can be made of any biologically adaptable or compatible materials. Materials considered acceptable for biological implantation are well known and include, but are not limited to, stainless steel, titanium, tantalum, combination metallic alloys, various plastics, resins, ceramics, biologically absorbable materials and the like. Any components may be also coated/made with nanotube materials to further impart unique mechanical or biological properties. In addition, any components may be also coated/made with osteo-conductive (such as deminerized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation. Further, any surface may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening. Lastly, any disclosed devices or any of its components can also be entirely or partially made of a shape memory material or other deformable/malleable material.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. An orthopedic implant adapted to resist anterior movement between a first vertebral bone and a second vertebral bone in a horizontal plane, comprising:
a first member that is adapted to affix onto the first bone;
a second member that is adapted to abut a segment of the second bone and that can move relative to the first member;
at least one flexible rotational articulation member that is contained within the implant and that provides at least a portion of the movement between the first and second members, the articulation member having:
a first hollow cylindrical member comprised of an outer surface with a defined radius from a longitudinal central axis, an inner surface with a defined radius from a longitudinal central axis, a thickness and an internal cavity that is contained within the inner surface, wherein at least one cylindrical tab extends from one end of the first follow cylindrical member wherein the tab circumferentially extends less than one hundred and eighty degrees around the longitudinal central axis;
a second hollow cylindrical member comprised of an outer surface with a defined radius from the longitudinal central axis, an inner surface with a defined radius from the longitudinal central axis, a thickness and an internal cavity that is contained within the inner surface of the second hollow cylindrical member, wherein at least one cylindrical tab extends from one end of the second hollow cylindrical member wherein the tab circumferentially extends less than one hundred and eighty degrees around its longitudinal central axis;
wherein the first and second members are axially aligned and wherein one tab of the first member is positioned within the internal cavity of the second member and one tab of the second member is positioned within the internal cavity of the first member;
wherein at least one flexible element connects the inner surface of the tab member of the first member with the inner surface of the second member and at least one flexible element connects the inner surface of the tab member of the second member with the inner surface of the first member so that said first and second may smoothly rotate relative to one another about the central longitudinal axis.

2. An orthopedic implant adapted to resist anterior movement between a first vertebral bone and a second vertebral bone in a horizontal plane, comprising:
a first member that is adapted to affix onto the first bone, wherein the first member contains a cavity that is adapted to contain a bone graft material and fuse with the first bone;
a second member that is adapted to abut a segment of the second bone, but not rigidly affix onto it;
wherein the implant permits relative movement between the first and second vertebral bones.

3. A method for resisting translation of a first vertebral bone relative to a second vertebral bone in a horizontal plane, comprising:
rigidly affixing an implant onto the first bone, wherein the implant contains a cavity that is adapted to contain a bone graft material and to fuse with the first bone;
placing a segment of the implant posterior to a posterior surface of the second vertebral bone; and
positioning the implant segment so that it abuts, but does not rigidly affix, onto the posterior surface of the second vertebral bone;
wherein the implant permits relative movement between the first and second vertebral bones.
